SEQUENCE LISTING (92 sequences)

SEQ ID NO: 1 - protein, 107 aa, Artificial Sequence
  source: /note=“Description of Artificial Sequence Synthetic polypeptide”
DIVMTQSQKFMSTSVGDRVSLTCKASQNVGTAVAWYQQKPGQSPKLLIYSASNRYTGVPDRFTGSGSGTDFTLTISNMQSEDLADYFCQQYSSYPLTFGAGTKVEIK

SEQ ID NO: 2 - protein, 214 aa, Artificial Sequence
  source: /note=“Description of Artificial Sequence Synthetic polypeptide”
DIVMTQSQKFMSTSVGDRVSLTCKASQNVGTAVAWYQQKPGQSPKLLIYSASNRYTGVPDRFTGSGSGTDFTLTISNMQSEDLADYFCQQYSSYPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 107 aa, Artificial Sequence
  source: /note=“Description of Artificial Sequence Synthetic polypeptide”
DIQMTQSPSSLSASVGDRVTITCKASQNVGTAVAWYQQKPGKAPKLLIYSASNRYTGVPSRFSGSGSGTDFTLTISSLQPEDFATYYCQQYSSYPLTFGQGTKVEIK

SEQ ID NO: 4 - protein, 214 aa, Artificial Sequence
  source: /note=“Description of Artificial Sequence Synthetic polypeptide”
DIQMTQSPSSLSASVGDRVTITCKASQNVGTAVAWYQQKPGKAPKLLIYSASNRYTGVPSRFSGSGSGTDFTLTISSLQPEDFATYYCQQYSSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 107 aa, Artificial Sequence
  source: /note=“Description of Artificial Sequence Synthetic polypeptide”
DIQMTQSPSSLSASVGDRVTITCKASQNVGTAVAWYQQKPGKSPKLLIYSASNRYTGVPSRFSGSGSGTDFTLTISSLQPEDFATYYCQQYSSYPLTFGQGTKVEIK

SEQ ID NO: 6 - protein, 214 aa, Artificial Sequence
  source: /note=“Description of Artificial Sequence Synthetic polypeptide”
DIQMTQSPSSLSASVGDRVTITCKASQNVGTAVAWYQQKPGKSPKLLIYSASNRYTGVPSRFSGSGSGTDFTLTISSLQPEDFATYYCQQYSSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 107 aa, Artificial Sequence
  source: /note=“Description of Artificial Sequence Synthetic polypeptide”
DIQMTQSPSSLSASVGDRVTITCKASQNVGTAVAWYQQKPGKSPKLLIYSASNRYTGVPSRFSGSGSGTDFTLTISSLQPEDFATYFCQQYSSYPLTFGQGTKVEIK

SEQ ID NO: 8 - protein, 214 aa, Artificial Sequence
  source: /note=“Description of Artificial Sequence Synthetic polypeptide”
DIQMTQSPSSLSASVGDRVTITCKASQNVGTAVAWYQQKPGKSPKLLIYSASNRYTGVPSRFSGSGSGTDFTLTISSLQPEDFATYFCQQYSSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 11 aa, Artificial Sequence
  source: /note=“Description of Artificial Sequence Synthetic peptide”
KASQNVGTAVA

SEQ ID NO: 10 - protein, 7 aa, Artificial Sequence
  source: /note=“Description of Artificial Sequence Synthetic peptide”
SASNRYT

SEQ ID NO: 11 - protein, 9 aa, Artificial Sequence
  source: /note=“Description of Artificial Sequence Synthetic peptide”
QQYSSYPLT

SEQ ID NO: 12 - protein, 122 aa, Artificial Sequence
  source: /note=“Description of Artificial Sequence Synthetic polypeptide”
QLTLKESGPGILKPSQTLSLTCSLSGFSLSTSGVGVGWFRQPSGKGLEWLALIWWDDDKYYNPSLKSQLSISKDFSRNQVFLKISNVDIADTATYYCARRDPFGYDNAMGYWGQGTSVTVSS

SEQ ID NO: 13 - protein, 452 aa, Artificial Sequence
  source: /note=“Description of Artificial Sequence Synthetic polypeptide”
QLTLKESGPGILKPSQTLSLTCSLSGFSLSTSGVGVGWFRQPSGKGLEWLALIWWDDDKYYNPSLKSQLSISKDFSRNQVFLKISNVDIADTATYYCARRDPFGYDNAMGYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 122 aa, Artificial Sequence
  source: /note=“Description of Artificial Sequence Synthetic polypeptide”
EVQLVESGPALVKPTQTLTLTCTFSGFSLSTSGVGVGWIRQPPGKALEWLALIWWDDDKYYNPSLKSRLTISKDTSKNQVVLTMTNMDPVDTATYYCARRDPFGYDNAMGYWGQGTLVTVSS

SEQ ID NO: 15 - protein, 451 aa, Artificial Sequence
  source: /note=“Description of Artificial Sequence Synthetic polypeptide”
EVQLVESGPALVKPTQTLTLTCTFSGFSLSTSGVGVGWIRQPPGKALEWLALIWWDDDKYYNPSLKSRLTISKDTSKNQVVLTMTNMDPVDTATYYCARRDPFGYDNAMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSWQQGNVFSCSVMHEALHNHYTQKSLSLSPGK

SEQ ID NO: 16 - protein, 122 aa, Artificial Sequence
  source: /note=“Description of Artificial Sequence Synthetic polypeptide”
ELQLVESGPALVKPTQTLTLTCTLSGFSLSTSGVGVGWFRQPPGKALEWLALIWWDDDKYYNPSLKSRLTISKDTSKNQVVLTMTNMDPVDTATYYCARRDPFGYDNAMGYWGQGTLVTVSS

SEQ ID NO: 17 - protein, 451 aa, Artificial Sequence
  source: /note=“Description of Artificial Sequence Synthetic polypeptide”
ELQLVESGPALVKPTQTLTLTCTLSGFSLSTSGVGVGWFRQPPGKALEWLALIWWDDDKYYNPSLKSRLTISKDTSKNQVVLTMTNMDPVDTATYYCARRDPFGYDNAMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSWQQGNVFSCSVMHEALHNHYTQKSLSLSPGK

SEQ ID NO: 18 - protein, 122 aa, Artificial Sequence
  source: /note=“Description of Artificial Sequence Synthetic polypeptide”
ELQLVESGPALVKPTQTLTLTCTLSGFSLSTSGVGVGWFRQPPGKALEWLALIWWDDDKYYNPSLKSRLTISKDFSRNQVVLTMTNMDPVDTATYYCARRDPFGYDNAMGYWGQGTLVTVSS

SEQ ID NO: 19 - protein, 451 aa, Artificial Sequence
  source: /note=“Description of Artificial Sequence Synthetic polypeptide”
ELQLVESGPALVKPTQTLTLTCTLSGFSLSTSGVGVGWFRQPPGKALEWLALIWWDDDKYYNPSLKSRLTISKDFSRNQVVLTMTNMDPVDTATYYCARRDPFGYDNAMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSWQQGNVFSCSVMHEALHNHYTQKSLSLSPGK

SEQ ID NO: 20 - protein, 122 aa, Artificial Sequence
  source: /note=“Description of Artificial Sequence Synthetic polypeptide”
ELQLVESGPALVKPTQTLTLTCTLSGFSLSTSGVGVGWFRQPPGKALEWLALIWWDDDKYYNPSLKSQLSISKDFSRNQVVLTMTNMDPVDTATYYCARRDPFGYDNAMGYWGQGTLVTVSS

SEQ ID NO: 21 - protein, 451 aa, Artificial Sequence
  source: /note=“Description of Artificial Sequence Synthetic polypeptide”
ELQLVESGPALVKPTQTLTLTCTLSGFSLSTSGVGVGWFRQPPGKALEWLALIWWDDDKYYNPSLKSQLSISKDFSRNQVVLTMTNMDPVDTATYYCARRDPFGYDNAMGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSWQQGNVFSCSVMHEALHNHYTQKSLSLSPGK

SEQ ID NO: 22 - protein, 12 aa, Artificial Sequence
  source: /note=“Description of Artificial Sequence Synthetic peptide”
GFSLSTSGVGVG

SEQ ID NO: 23 - protein, 16 aa, Artificial Sequence
  source: /note=“Description of Artificial Sequence Synthetic peptide”
LIWWDDDKYYNPSLKS

SEQ ID NO: 24 - protein, 14 aa, Artificial Sequence
  source: /note=“Description of Artificial Sequence Synthetic peptide”
ARRDPFGYDNAMGY

SEQ ID NO: 25 - protein, 107 aa, Artificial Sequence
  source: /note=“Description of Artificial Sequence Synthetic polypeptide”
DIVMTQSPASLSVSVGETVSITCRASENIYSSLAWYQQKQGKSPQLLVYSATILADGVPSRFSGSGSGTQYSLKINSLQSEDFGTYYCQHFWGTPYTFGGGTKVEIK

SEQ ID NO: 26 - protein, 214 aa, Artificial Sequence
  source: /note=“Description of Artificial Sequence Synthetic polypeptide”
DIVMTQSPASLSVSVGETVSITCRASENIYSSLAWYQQKQGKSPQLLVYSATILADGVPSRFSGSGSGTQYSLKINSLQSEDFGTYYCQHFWGTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 107 aa, Artificial Sequence
  source: /note=“Description of Artificial Sequence Synthetic polypeptide”
DIQMTQSPSSLSASVGDRVTITCRASENIYSSLAWYQQKPGKAPKLLIYSATILADGVPSRFSGSGSGTDFTLTISSLQPEDFATYYCQHFWGTPYTFGQGTKVEIK

SEQ ID NO: 28 - protein, 214 aa, Artificial Sequence
  source: /note=“Description of Artificial Sequence Synthetic polypeptide”
DIQMTQSPSSLSASVGDRVTITCRASENIYSSLAWYQQKPGKAPKLLIYSATILADGVPSRFSGSGSGTDFTLTISSLQPEDFATYYCQHFWGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 107 aa, Artificial Sequence
  source: /note=“Description of Artificial Sequence Synthetic polypeptide”
DIQMTQSPSSLSASVGDRVTITCRASENIYSSLAWYQQKPGKSPKLLIYSATILADGVPSRFSGSGSGTDFTLTISSLQPEDFATYYCQHFWGTPYTFGQGTKVEIK

SEQ ID NO: 30 - protein, 214 aa, Artificial Sequence
  source: /note=“Description of Artificial Sequence Synthetic polypeptide”
DIQMTQSPSSLSASVGDRVTITCRASENIYSSLAWYQQKPGKSPKLLIYSATILADGVPSRFSGSGSGTDFTLTISSLQPEDFATYYCQHFWGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 107 aa, Artificial Sequence
  source: /note=“Description of Artificial Sequence Synthetic polypeptide”
DIQMTQSPSSLSASVGDRVTITCRASENIYSSLAWYQQKPGKAPKLLVYSATILADGVPSRFSGSGSGTDFTLTISSLQPEDFATYYCQHFWGTPYTFGQGTKVEIK

SEQ ID NO: 32 - protein, 214 aa, Artificial Sequence
  source: /note=“Description of Artificial Sequence Synthetic polypeptide”
DIQMTQSPSSLSASVGDRVTITCRASENIYSSLAWYQQKPGKAPKLLVYSATILADGVPSRFSGSGSGTDFTLTISSLQPEDFATYYCQHFWGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 107 aa, Artificial Sequence
  source: /note=“Description of Artificial Sequence Synthetic polypeptide”
DIQMTQSPSSLSASVGDRVTITCRASENIYSSLAWYQQKPGKSPKLLVYSATILADGVPSRFSGSGSGTDFTLTISSLQPEDFATYYCQHFWGTPYTFGQGTKVEIK

SEQ ID NO: 34 - protein, 214 aa, Artificial Sequence
  source: /note=“Description of Artificial Sequence Synthetic polypeptide”
DIQMTQSPSSLSASVGDRVTITCRASENIYSSLAWYQQKPGKSPKLLVYSATILADGVPSRFSGSGSGTDFTLTISSLQPEDFATYYCQHFWGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 107 aa, Artificial Sequence
  source: /note=“Description of Artificial Sequence Synthetic polypeptide”
DIQMTQSPSSLSASVGDRVTITCRASENIYSSLAWYQQKPGKSPKLLVYSATILADGVPSRFSGSGSGTDYTLTISSLQPEDFATYYCQHFWGTPYTFGQGTKVEIK

SEQ ID NO: 36 - protein, 214 aa, Artificial Sequence
  source: /note=“Description of Artificial Sequence Synthetic polypeptide”
DIQMTQSPSSLSASVGDRVTITCRASENIYSSLAWYQQKPGKSPKLLVYSATILADGVPSRFSGSGSGTDYTLTISSLQPEDFATYYCQHFWGTPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 11 aa, Artificial Sequence
  source: /note=“Description of Artificial Sequence Synthetic peptide”
RASENIYSSLA

SEQ ID NO: 38 - protein, 7 aa, Artificial Sequence
  source: /note=“Description of Artificial Sequence Synthetic peptide”
SATILAD

SEQ ID NO: 39 - protein, 9 aa, Artificial Sequence
  source: /note=“Description of Artificial Sequence Synthetic peptide”
QHFWGTPYT

SEQ ID NO: 40 - protein, 121 aa, Artificial Sequence
  source: /note=“Description of Artificial Sequence Synthetic polypeptide”
EVQLVESGGGLMQPGGSMKLSCVASGFTFSNYWMNWVRQSPEKGLEWVAEIRLKSNNYATHYAESVKGRFTISRDDSKRSVYLQMNTLRAEDTGIYYCTRGNGNYRAMDYWGQGTSVTVSS

SEQ ID NO: 41 - protein, 451 aa, Artificial Sequence
  source: /note=“Description of Artificial Sequence Synthetic polypeptide”
EVQLVESGGGLMQPGGSMKLSCVASGFTFSNYWMNWVRQSPEKGLEWVAEIRLKSNNYATHYAESVKGRFTISRDDSKRSVYLQMNTLRAEDTGIYYCTRGNGNYRAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 121 aa, Artificial Sequence
  source: /note=“Description of Artificial Sequence Synthetic polypeptide”
EVQLVESGGGLVQPGGSLRLSCAASGFTFSNYWMNWVRQAPGKGLEWVGEIRLKSNNYATHYAESVKGRFTISRDNSKNTLYLQMNSLRAEDTAVYYCTRGNGNYRAMDYWGQGTLVTVSS

SEQ ID NO: 43 - protein, 451 aa, Artificial Sequence
  source: /note=“Description of Artificial Sequence Synthetic polypeptide”
EVQLVESGGGLVQPGGSLRLSCAASGFTFSNYWMNWVRQAPGKGLEWVGEIRLKSNNYATHYAESVKGRFTISRDNSKNTLYLQMNSLRAEDTAVYYCTRGNGNYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 121 aa, Artificial Sequence
  source: /note=“Description of Artificial Sequence Synthetic polypeptide”
EVQLVESGGGLVQPGGSLRLSCAASGFTFSNYWMNWVRQAPGKGLEWVAEIRLKSNNYATHYAESVKGRFTISRDNSKNTLYLQMNSLRAEDTAVYYCTRGNGNYRAMDYWGQGTLVTVSS

SEQ ID NO: 45 - protein, 451 aa, Artificial Sequence
  source: /note=“Description of Artificial Sequence Synthetic polypeptide”
EVQLVESGGGLVQPGGSLRLSCAASGFTFSNYWMNWVRQAPGKGLEWVAEIRLKSNNYATHYAESVKGRFTISRDNSKNTLYLQMNSLRAEDTAVYYCTRGNGNYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 121 aa, Artificial Sequence
  source: /note=“Description of Artificial Sequence Synthetic polypeptide”
EVQLVESGGGLVQPGGSLRLSCAASGFTFSNYWMNWVRQAPGKGLEWVGEIRLKSNNYATHYAESVKGRFTISRDNSKNTVYLQMNSLRAEDTAVYYCTRGNGNYRAMDYWGQGTLVTVSS

SEQ ID NO: 47 - protein, 451 aa, Artificial Sequence
  source: /note=“Description of Artificial Sequence Synthetic polypeptide”
EVQLVESGGGLVQPGGSLRLSCAASGFTFSNYWMNWVRQAPGKGLEWVGEIRLKSNNYATHYAESVKGRFTISRDNSKNTVYLQMNSLRAEDTAVYYCTRGNGNYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 121 aa, Artificial Sequence
  source: /note=“Description of Artificial Sequence Synthetic polypeptide”
EVQLVESGGGLVQPGGSLRLSCAASGFTFSNYWMNWVRQAPGKGLEWVAEIRLKSNNYATHYAESVKGRFTISRDNSKNTVYLQMNSLRAEDTAVYYCTRGNGNYRAMDYWGQGTLVTVSS

SEQ ID NO: 49 - protein, 451 aa, Artificial Sequence
  source: /note=“Description of Artificial Sequence Synthetic polypeptide”
EVQLVESGGGLVQPGGSLRLSCAASGFTFSNYWMNWVRQAPGKGLEWVAEIRLKSNNYATHYAESVKGRFTISRDNSKNTVYLQMNSLRAEDTAVYYCTRGNGNYRAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 10 aa, Artificial Sequence
  source: /note=“Description of Artificial Sequence Synthetic peptide”
GFTFSNYWMN

SEQ ID NO: 51 - protein, 19 aa, Artificial Sequence
  source: /note=“Description of Artificial Sequence Synthetic peptide”
EIRLKSNNYATHYAESVKG

SEQ ID NO: 52 - protein, 12 aa, Artificial Sequence
  source: /note=“Description of Artificial Sequence Synthetic peptide”
TRGNGNYRAMDY

SEQ ID NO: 53 - protein, 107 aa, Artificial Sequence
  source: /note=“Description of Artificial Sequence Synthetic polypeptide”
DIVLTQSPASLSASVGETVTITCRASENIYSYLAWYQQKQGKSPQLLVYNAKTLIEGVPSRFSGSGSGTQFSLKINSLQPEDFGSYFCQHHFGTPFTFGQGTKVEIK

SEQ ID NO: 54 - protein, 214 aa, Artificial Sequence
  source: /note=“Description of Artificial Sequence Synthetic polypeptide”
DIVLTQSPASLSASVGETVTITCRASENIYSYLAWYQQKQGKSPQLLVYNAKTLIEGVPSRFSGSGSGTQFSLKINSLQPEDFGSYFCQHHFGTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 107 aa, Artificial Sequence
  source: /note=“Description of Artificial Sequence Synthetic polypeptide”
DIQMTQSPSSLSASVGDRVTITCRASENIYSYLAWYQQKPGKAPKLLIYNAKTLIEGVPSRFSGSGSGTDFTLTISSLQPEDFATYYCQHHFGTPFTFGQGTKVEIK

SEQ ID NO: 56 - protein, 214 aa, Artificial Sequence
  source: /note=“Description of Artificial Sequence Synthetic polypeptide”
DIQMTQSPSSLSASVGDRVTITCRASENIYSYLAWYQQKPGKAPKLLIYNAKTLIEGVPSRFSGSGSGTDFTLTISSLQPEDFATYYCQHHFGTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 107 aa, Artificial Sequence
  source: /note=“Description of Artificial Sequence Synthetic polypeptide”
DIQMTQSPSSLSASVGDRVTITCRASENIYSYLAWYQQKPGKSPKLLIYNAKTLIEGVPSRFSGSGSGTDFTLTISSLQPEDFATYYCQHHFGTPFTFGQGTKVEIK

SEQ ID NO: 58 - protein, 214 aa, Artificial Sequence
  source: /note=“Description of Artificial Sequence Synthetic polypeptide”
DIQMTQSPSSLSASVGDRVTITCRASENIYSYLAWYQQKPGKSPKLLIYNAKTLIEGVPSRFSGSGSGTDFTLTISSLQPEDFATYYCQHHFGTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 107 aa, Artificial Sequence
  source: /note=“Description of Artificial Sequence Synthetic polypeptide”
DIQMTQSPSSLSASVGDRVTITCRASENIYSYLAWYQQKPGKAPKLLVYNAKTLIEGVPSRFSGSGSGTDFTLTISSLQPEDFATYYCQHHFGTPFTFGQGTKVEIK

SEQ ID NO: 60 - protein, 214 aa, Artificial Sequence
  source: /note=“Description of Artificial Sequence Synthetic polypeptide”
DIQMTQSPSSLSASVGDRVTITCRASENIYSYLAWYQQKPGKAPKLLVYNAKTLIEGVPSRFSGSGSGTDFTLTISSLQPEDFATYYCQHHFGTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 107 aa, Artificial Sequence
  source: /note=“Description of Artificial Sequence Synthetic polypeptide”
DIQMTQSPSSLSASVGDRVTITCRASENIYSYLAWYQQKPGKSPKLLVYNAKTLIEGVPSRFSGSGSGTDFTLTISSLQPEDFATYYCQHHFGTPFTFGQGTKVEIK

SEQ ID NO: 62 - protein, 214 aa, Artificial Sequence
  source: /note=“Description of Artificial Sequence Synthetic polypeptide”
DIQMTQSPSSLSASVGDRVTITCRASENIYSYLAWYQQKPGKSPKLLVYNAKTLIEGVPSRFSGSGSGTDFTLTISSLQPEDFATYYCQHHFGTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 107 aa, Artificial Sequence
  source: /note=“Description of Artificial Sequence Synthetic polypeptide”
DIQMTQSPSSLSASVGDRVTITCRASENIYSYLAWYQQKPGKSPKLLVYNAKTLIEGVPSRFSGSGSGTDFTLTISSLQPEDFATYFCQHHFGTPFTFGQGTKVEIK

SEQ ID NO: 64 - protein, 214 aa, Artificial Sequence
  source: /note=“Description of Artificial Sequence Synthetic polypeptide”
DIQMTQSPSSLSASVGDRVTITCRASENIYSYLAWYQQKPGKSPKLLVYNAKTLIEGVPSRFSGSGSGTDFTLTISSLQPEDFATYFCQHHFGTPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 11 aa, Artificial Sequence
  source: /note=“Description of Artificial Sequence Synthetic peptide”
RASENIYSYLA

SEQ ID NO: 66 - protein, 7 aa, Artificial Sequence
  source: /note=“Description of Artificial Sequence Synthetic peptide”
NAKTLIE

SEQ ID NO: 67 - protein, 9 aa, Artificial Sequence
  source: /note=“Description of Artificial Sequence Synthetic peptide”
QHHFGTPFT

SEQ ID NO: 68 - protein, 120 aa, Artificial Sequence
  source: /note=“Description of Artificial Sequence Synthetic polypeptide”
EVQLQESGPELEMPGASVKISCKASGSSFTGFSMNWVKQSNGKSLEWIGNIDTYYGGTTYNQKFKGKATLTVDKSSSTAYMQLKSLTSEDTSAVYYCARSAYYGSTFAYWGQGTSVTVSS

SEQ ID NO: 69 - protein, 450 aa, Artificial Sequence
  source: /note=“Description of Artificial Sequence Synthetic polypeptide”
EVQLQESGPELEMPGASVKISCKASGSSFTGFSMNWVKQSNGKSLEWIGNIDTYYGGTTYNQKFKGKATLTVDKSSSTAYMQLKSLTSEDTSAVYYCARSAYYGSTFAYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 120 aa, Artificial Sequence
  source: /note=“Description of Artificial Sequence Synthetic polypeptide”
EVQLVQSGAEVKKPGASVKVSCKASGSSFTGFSMNWVRQAPGQGLEWIGNIDTYYGGTTYNQKFKGRVTITRDTSTSTAYLELSSLRSEDTAVYYCCARSAYYGSTFAYWGQGTLVTVSS

SEQ ID NO: 71 - protein, 450 aa, Artificial Sequence
  source: /note=“Description of Artificial Sequence Synthetic polypeptide”
EVQLVQSGAEVKKPGASVKVSCKASGSSFTGFSMNWVRQAPGQGLEWIGNIDTYYGGTTYNQKFKGRVTITRDTSTSTAYLELSSLRSEDTAVYYCCARSAYYGSTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 120 aa, Artificial Sequence
  source: /note=“Description of Artificial Sequence Synthetic polypeptide”
EVQLVQSGAEVKKPGASVKVSCKASGSSFTGFSMNWVRQAPGQGLEWIGNIDTYYGGTTYNQKFKGRVTITVDTSTSTAYLELSSLRSEDTAVYYCCARSAYYGSTFAYWGQGTLVTVSS

SEQ ID NO: 73 - protein, 450 aa, Artificial Sequence
  source: /note=“Description of Artificial Sequence Synthetic polypeptide”
EVQLVQSGAEVKKPGASVKVSCKASGSSFTGFSMNWVRQAPGQGLEWIGNIDTYYGGTTYNQKFKGRVTITVDTSTSTAYLELSSLRSEDTAVYYCCARSAYYGSTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 120 aa, Artificial Sequence
  source: /note=“Description of Artificial Sequence Synthetic polypeptide”
EVQLVQSGAEVKKPGASVKVSCKASGSSFTGFSMNWVRQAPGQGLEWIGNIDTYYGGTTYNQKFKGRATLTVDTSTSTAYLELSSLRSEDTAVYYCCARSAYYGSTFAYWGQGTLVTVSS

SEQ ID NO: 75 - protein, 450 aa, Artificial Sequence
  source: /note=“Description of Artificial Sequence Synthetic polypeptide”
EVQLVQSGAEVKKPGASVKVSCKASGSSFTGFSMNWVRQAPGQGLEWIGNIDTYYGGTTYNQKFKGRATLTVDTSTSTAYLELSSLRSEDTAVYYCCARSAYYGSTFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 10 aa, Artificial Sequence
  source: /note=“Description of Artificial Sequence Synthetic peptide”
GSSFTGFSMN

SEQ ID NO: 77 - protein, 17 aa, Artificial Sequence
  source: /note=“Description of Artificial Sequence Synthetic peptide”
NIDTYYGGTTYNQKFKG

SEQ ID NO: 78 - protein, 13 aa, Artificial Sequence
  source: /note=“Description of Artificial Sequence Synthetic peptide”
CARSAYYGSTFAY

SEQ ID NO: 79 - protein, 676 aa, Zaire ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 80 - protein, 676 aa, Sudan ebolavirus
MEGLSLLQLPRDKFRKSSFFVWVIILFQKAFSMPLGVVTNSTLEVTEIDQLVCKDHLASTDQLKSVGLNLEGSGVSTDIPSATKRWGFRSGVPPQVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAANYTENTSSYYATSYLEYEIENFGAQHSTTLFKINNNTFVLLDRPHTPQFLFQLNDTIQLHQQLSNTTGKLIWTLDANINADIGEWAFWENKKNLSEQLRGEELSFETLSLNETEDDDATSSRTTKGRISDRATRKYSDLVPKDSPGMVSLHVPEGETTLPSQNSTEGRRVDVNTQETITETTATIIGTNGNNMQISTIGTGLSSSQILSSSPTMAPSPETQTSTTYTPKLPVMTTEESTTPPRNSPGSTTEAPTLTTPENITTAVKTVWPQESTSNGLITSTVTGILGSLGLRKRSRRQVNTRATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 81 - protein, 677 aa, Reston ebolavirus
MGSGYQLLQLPRERFRKTSFLVWVIILFQRAISMPLGIVTNSTLKATEIDQLVCRDKLSSTSQLKSVGLNLEGNGIATDVPSATKRWGFRSGVPPKVVSYEAGEWAENCYNLEIKKSDGSECLPLPPDGVRGFPRCRYVHKVQGTGPCPGDLAFHKNGAFFLYDRLASTVIYRGTTFAEGVVAFLILSEPKKHFWKATPAHEPVNTTDDSTSYYMTLTLSYEMSNFGGNESNTLFKVDNHTYVQLDRPHTPQFLVQLNETLRRNNRLSNSTGRLTWTLDPKIEPDVGEWAFWETKKNFSQQLHGENLHFQILSTHTNNSSDQSPAGTVQGKISYHPPANNSELVPTDSPPVVSVLTAGRTEEMSTQGLTNGETITGFTANPMTTTIAPSPTMTSEVDNNVPSEQPNNTASIEDSPPSASNETIYHSEMDPIQGSNNSAQSPQTKTTPAPTTSPMTQDPQETANSSKPGTSPGSAAGPSQPGLTINTVSKVADSLSPTRKQKRSVRQNTANKCNPDLYYWTAVDEGAAVGLAWIPYFGPAAEGIYIEGVMHNQNGLICGLRQLANETTQALQLFLRATTELRTYSLLNRKAIDFLLQRWGGTCRILGPSCCIEPHDWTKNITDEINQIKHDFIDNPLPDHGDDLNLWTGWRQWIPAGIGIIGVIIAIIALLCICKILC

SEQ ID NO: 82 - protein, 676 aa, Sudan ebolavirus
MGGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGEWAFWENKKNLSEQLRGEELSFEALSLNETEDDDAASSRITKGRISDRATRKYSDLVPKNSPGMVPLHIPEGETTLPSQNSTEGRRVGVNTQETITETAATIIGTNGNHMQISTIGIRPSSSQIPSSSPTTAPSPEAQTPTTHTSGPSVMATEEPTTPPGSSPGPTTEAPTLTTPENITTAVKTVLPQESTSNGLITSTVTGILGSLGLRKRSRRQTNTKATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 83 - protein, 676 aa, Zaire ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETRYTSGKRSNTTGKLIWKVNPEIDTTIGEWAFWETKKNLTRKIRSEELSFTAVSNRAKNISGQSPARTSSDPGTNTTTEDHKIMASENSSAMVQVHSQGREAAVSHLTTLATISTSLRPPITKPGPDNSTHNTPVYKLDISEATQVEQHHRRTDNASTTSDTPPATTAAGPLKAENTNTSKGTDLLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 84 - protein, 677 aa, Reston ebolavirus
MGSGYQLLQLPRERFRKTSFLVWVIILFQRAISMPLGIVTNSTLKATEIDQLVCRDKLSSTSQLKSVGLNLEGNGIATDVPSATKRWGFRSGVPPKVVSYEAGEWAENCYNLEIKKSDGSECLPLPPDGVRGFPRCRYVHKVQGTGPCPGDLAFHKNGAFFLYDRLASTVIYRGTTFAEGVIAFLILSEPKKHFWKATPAHEPVNTTDDSTSYYMTLTLSYEMSNFGGEESNTLFKVDNHTYVQLDRPHTPQFLVQLNETLRRNNRLSNSTGRLTWTVDPKIEPDVGEWAFWETKKNFSQQLHGENLHFQILSTHTNNSSDQSPAGTVQGKISYHPPTNNSELVPTDSPPVVSVLTAGRTEEMSTQGLTNGETITGFTANPMTTTIAPSPTMTSEVDNNVPSEQPNNTASIEDSPPSASNETIDHSEMNSIQGSNNSAQSPQTKATPAPTASPMTLDPQETANISKPGTSPGSAAGPSQPGLTINTISKVADSLSPTRKQKRSVRQNTANKCNPDLHYWTAVDEGAAAGLAWIPYFGPAAEGIYIEGVMHNQNGLICGLRQLANETTQALQLFLRATTELRTYSLLNRKAIDFLLQRWGGTCRILGPSCCIEPHDWTKNITDEINQIKHDFIDNPLPDHGDDLNLWTGWRQWIPAGIGIIGVIIAIIALLCICKILC

SEQ ID NO: 85 - protein, 676 aa, Zaire ebolavirus
MGVTGILQLPRDRFKRTSFFLWVIILFQRTFSIPLGVIHNSTLQVNDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYTEGLMHNQNGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 86 - protein, 677 aa, Reston ebolavirus
MGSGYQLLQLPRERFRKTSFLVWVIILFQRAISMPLGIVTNSTLKATEIDQLVCRDKLSSTSQLKSVGLNLEGNGIATDVPSATKRWGFRSGVPPKVVSYEAGEWAENCYNLEIKKSDGSECLPLPPDGVRGFPRCRYVHKVQGTGPCPGDLAFHKNGAFFLYDRLASTVIYRGTTFTEGVVAFLILSEPKKHFWKATPAHEPVNTTDDSTSYYMTLTLSYEMSNFGGKESNTLFKVDNHTYVQLDRPHTPQFLVQLNETLRRNNRLSNSTGRLTWTLDPKIEPDVGEWAFWETKKNFSQQLHGENLHFQILSTHTNNSSDQSPAGTVQGKISYHPPTNNSELVPTDSPPVVSVLTAGRTEEMSTQGLTNGETITGFTANPMTTTIAPSPTMTSEVDNNVPSEQPNNTASIEDSPPSASNETIDHSEMNPIQGSNNSAQSPQTKTTPAPTASPMTQDPQETANSSKLGTSPGSAAEPSQPGFTINTVSKVADSLSPTRKQKRSVRQNTANKCNPDLHYWTAVDEGAAVGLAWIPYFGPAAEGIYIEGVMHNQNGLICGLRQLANETTQALQLFLRATTELRTYSLLNRKAIDFLLQRWGGTCRILGPSCCIEPHDWTKNITDEINQIKHDFIDNPLPDHGDDLNLWTGWRQWIPAGIGIIGVIIAIIALLCICKILC

SEQ ID NO: 87 - protein, 676 aa, Sudan ebolavirus
MEGLSLLQLPRDKFRKSSFFVWVIILFQKAFSMPLGVVTNSTLEVTEIDQLVCKDHLASTDQLKSVGLNLEGSGVSTDIPSATKRWGFRSGVPPQVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAANYTENTSSYYATSYLEYEIENFGAQHSTTLFKINNNTFVLLDRPHTPQFLFQLNDTIQLHQQLSNTTGKLIWTLDANINADIGEWAFWENKKNLSEQLRGEELSFETLSLNETEDDDATSSRTTKGRISDRATRKYSDLVPKDSPGMVSLHVPEGETTLPSQNSTEGRRVDVNTQETITETTATIIGTNGNNMQISTIGTGLSSSQILSSSPTMAPSPETQTSTTYTPKLPVMTTEEPTTPPRNSPGSTTEAPTLTTPENITTAVKTVWAQESTSNGLITSTVTGILGSLGLRKRSRRQVNTRATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 88 - protein, 676 aa, Tai Forest ebolavirus
MGASGILQLPRERFRKTSFFVWVIILFHKVFSIPLGVVHNNTLQVSDIDKFVCRDKLSSTSQLKSVGLNLEGNGVATDVPTATKRWGFRAGVPPKVVNYEAGEWAENCYNLAIKKVDGSECLPEAPEGVRDFPRCRYVHKVSGTGPCPGGLAFHKEGAFFLYDRLASTIIYRGTTFAEGVIAFLILPKARKDFFQSPPLHEPANMTTDPSSYYHTTTINYVVDNFGTNTTEFLFQVDHLTYVQLEARFTPQFLVLLNETIYSDNRRSNTTGKLIWKINPTVDTSMGEWAFWENKKNFTKTLSSEELSFVPVPETQNQVLDTTATVSPPISAHNHAGEDHKELVSEDSTPVVQMQNIKGKDTMPTTVTGVPTTTPSPFPINARNTDHTKSFIGLEGPQEDHSTTQPAKTTSQPTNSTESTTLNPTSEPSSRGTGPSSPTVPNTTESHAELGKTTPTTLPEQHTAASAIPRAVHPDELSGPGFLTNTIRGVTNLLTGSRRKRRDVTPNTQPKCNPNLHYWTALDEGAAIGLAWIPYFGPAAEGIYTEGIMENQNGLICGLRQLANETTQALQLFLRATTELRTFSILNRKAIDFLLQRWGGTCHILGPDCCIEPQDWTKNITDKIDQIIHDFVDNNLPNQNDGSNWWTGWKQWVPAGIGITGVIIAIIALLCICKFML

SEQ ID NO: 89 - protein, 676 aa, Zaire ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTAVSNRAKNISGQSPARTSSDPGTNTTTEDHKIMASENSSAMVQVHSQGREAAVSHLTTLATISTSPQPPTTKPGPDNSTHNTPVYKLDISEATQVEQHHRRTDNDSTASDTPPATTAAGPLKAENTNTSKGTDLLDPATTTSPQNHSETAGNNNTHHQDTGEESASSGKLGLITNTIAGVAGLITGGRRARREAIVNAQPKCNPNLHYWTTQDEGAAIGLAWIPYFGPAAEGIYTEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 90 - protein, 676 aa, Bundibugyo virus
MVTSGILQLPRERFRKTSFFVWVIILFHKVFPIPLGVVHNNTLQVSDIDKLVCRDKLSSTSQLKSVGLNLEGNGVATDVPTATKRWGFRAGVPPKVVNYEAGEWAENCYNLDIKKADGSECLPEAPEGVRGFPRCRYVHKVSGTGPCPEGFAFHKEGAFFLYDRLASTIIYRSTTFSEGVVAFLILPKTKKDFFQSPPLHEPANMTTDPSSYYHTVTLNYVADNFGTNMTNFLFQVDHPTYVQLEPRFTPQFLVQLNETIYTNGRRSNTTGTLIWKVNPTVDTGVGEWAFWENKKNFTKTLSSEELSVILVPRAQDPGSNQKTKVTPTSFANNQTSKNHEDLVPKDPASVVQVRDLQRENTVPTSPLNTVPTTLIPDTMEEQTTSHYELPNISGNHQERNNTAHPETLANNPPDNTTPSTPPQDGERTSSHTTPSPRPVPTSTIHPTTRETQIPTTMITSHDTDSNRPNPIDISESTEPGLLTNTIRGVANLLTGSRRTRREITLRTQAKCNPNLHYWTTQDEGAAIGLAWIPYFGPAAEGIYTEGIMHNQNGLICGLRQLANETTQALQLFLRATTELRTFSILNRKAIDFLLQRWGGTCHILGPDCCIEPHDWTKNITDKIDQIIHDFIDKPLPDQTDNDNWWTGWRQWVPAGIGITGVIIAVIALLCICKFLL

SEQ ID NO: 91 - protein, 105 aa, Artificial Sequence
  source: /note=“Description of Artificial Sequence Synthetic polypeptide”
HNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLI

SEQ ID NO: 92 - protein, 15 aa, Artificial Sequence
  source: /note=“Description of Artificial Sequence Synthetic peptide”
REAIVNAQPKCNPNL

(19) United States
(12) Patent Application Publication
(10) Pub. No.: US 2018/0362620 A1
(43) Pub. Date: Dec. 20, 2018

(54) HUMANIZED ANTI-EBOLA VIRUS GLYCOPROTEIN ANTIBODIES AND METHODS OF USE

(71) Applicant: Genentech, Inc., South San Francisco, CA (US)

(72) Inventors: Mark Dennis, South San Francisco, CA (US); Mary Mathieu, South San Francisco, CA (US)

(73) Assignee: Genentech, Inc., South San Francisco, CA (US)

(21) Appl. No.: 15/821,062

(22) Filed: Nov. 22, 2017

Related U.S. Application Data
(63) Continuation of application No. PCT/US2016/034775, filed on May 27, 2016.
(60) Provisional application No. 62/168,096, filed on May 29, 2015.

(51) Int. Cl. C07K 16/10; A61K 47/68; A61K 45/06; A61K 39/42

(52) U.S. Cl. CPC C07K 16/10; A61K 47/6841; A61K 47/6803; A61K 45/06; C07K 2317/92; C07K 2317/24; C07K 2317/34; C07K 2317/41; A61K 39/42

(57) ABSTRACT

The present invention relates to anti-Ebola virus envelope glycoprotein antibodies. The invention further relates to diagnostic methods which utilize the anti-Ebola virus glycoprotein antibodies and to pharmaceutical compositions which employ these antibodies therapeutically and prophylactically.

### CROSS REFERENCE TO RELATED APPLICATION

This application is a continuation of International Application No. PCT/US2016/034775, having an international filing date of 27 May 2016, which claims the benefit of provisional U.S. Application No. 62/168,096 filed 29 May 2015, which are hereby incorporated by reference in its entirety.

### SEQUENCE LISTING

The instant application contains a Sequence Listing which has been submitted electronically in ASCII format and is hereby incorporated by reference in its entirety. Said ASCII copy, created on Nov. 21, 2017, is named P32854_US_1_SL.txt and is 198,793 bytes in size.

### FIELD OF THE INVENTION

The present invention relates to antibodies to viral envelope glycoproteins, in particular, anti-Ebola virus envelope glycoprotein (GP) antibodies and methods of using the same.

### BACKGROUND

Ebola virus (EBOV) is the infectious agent causing Ebola virus disease (EVD). Ebola viruses belong to the genus Ebolavirus of the Filoviridae family. Ebola virus causes severe hemorrhagic fever in humans and non-human primates. The only protein present on the surface of the virus is the Ebola virus envelope glycoprotein (GP).

Currently, there are no licensed vaccines or treatments against Ebola virus. Over the past decade, several experimental strategies have shown promise in treating EBOV-challenged non-human primates after infection (see, e.g., Qui et al. (2014) Nature 514:47-53). To date, only antibody-based therapeutic approaches have demonstrated substantial benefits in non-human primates when administered greater than 24 hours past EBOV exposure. Exemplary antibodies include: MB-003 (consisting of human or human-mouse chimeric monoclonal antibodies c13C6, h13F6, and c6D8; see, e.g., Zeitlin et al. (2011) PNAS 108:20690-20694); ZMab (consisting of murine monoclonal antibodies m1H3, m2G4, and m4G7; see, e.g., Murin et al. (2014) PNAS 111:17182-17187; Audet et al. (2014) Science Reports 4:6881 pp. 1-7); and ZMapp (consisting of chimeric monoclonal antibodies c13C6, c2G4, and c4G7; see, e.g., U.S. Pat. No. 7,335,356; U.S. Pat. No. 8,513,381; Geisbert (2014) Nature 514:41-43; Zhang et al. (2014) Science China 57:987-988).

ZMapp rescued 100% of rhesus macaques when treatment was initiated up to 5 days post-EBOV challenge. Advanced disease, as indicated by elevated liver enzymes, mucosal hemorrhages, and generalized petechia, were reversed, leading to full recovery of the infected animals.

Despite these encouraging results showing efficacy in non-human primates, human clinical development of ZMapp has been limited due to various liabilities of current ZMapp that limit the ability to obtain sufficient, reliable, and manufacturable quantities of each of the three monoclonal antibodies contained within ZMapp for use in the clinical development of this therapeutic. In particular, production of sufficient amounts of each of the three monoclonal antibodies contained within the ZMapp cocktail has been limited, in part, due to its current manufacture in tobacco. Additionally, the three monoclonal antibodies contained within ZMapp are chimeric. Accordingly, a need exists to provide anti-EBOV monoclonal antibodies in sufficient and reliable amounts. The present invention addresses this need, in part, by providing various humanized anti-Ebola virus envelope glycoprotein monoclonal antibodies.

### SUMMARY

The invention provides anti-Ebola virus envelope glycoprotein antibodies and methods of using the same.

In some embodiments, the present invention provides an isolated, humanized antibody that binds to Ebola virus glycoprotein. In some embodiments, an anti-Ebola virus envelope glycoprotein antibody of the present invention binds to an epitope comprising the amino acid sequence of SEQ ID NO:91. In some embodiments, an anti-Ebola virus envelope glycoprotein antibody of the present invention binds to an epitope comprising the amino acid sequence of SEQ ID NO:92.

In some embodiments, the invention provides an anti-Ebola virus envelope glycoprotein antibody, wherein the antibody comprises (a) HVR-H1 comprising the amino acid sequence of SEQ ID NO:22; (b) HVR-H2 comprising the amino acid sequence of SEQ ID NO:23; and (c) HVR-H3 comprising the amino acid sequence of SEQ ID NO:24.

In some embodiments, the invention provides an anti-Ebola virus envelope glycoprotein antibody, wherein the antibody comprises (a) HVR-L1 comprising the amino acid sequence of SEQ ID NO:9; (b) HVR-L2 comprising the amino acid sequence of SEQ ID NO:10; and (c) HVR-L3 comprising the amino acid sequence of SEQ ID NO:11.

In some embodiments, the invention provides an anti-Ebola virus envelope glycoprotein antibody, wherein the antibody comprises (a) HVR-H1 comprising the amino acid sequence of SEQ ID NO:22; (b) HVR-H2 comprising the amino acid sequence of SEQ ID NO:23; (c) HVR-H3 comprising the amino acid sequence of SEQ ID NO:24; (d) HVR-L1 comprising the amino acid sequence of SEQ ID NO:9; (e) HVR-L2 comprising the amino acid sequence of SEQ ID NO:10; and (f) HVR-L3 comprising the amino acid sequence of SEQ ID NO:11.

In some embodiments, the invention provides an anti-Ebola virus envelope glycoprotein antibody, wherein the antibody comprises a heavy chain variable region, wherein the heavy chain variable region comprises an amino acid sequence selected from the group consisting of SEQ ID NOs:14, 16, 18, and 20.

In some embodiments, the invention provides an anti-Ebola virus envelope glycoprotein antibody, wherein the antibody comprises a light chain variable region, wherein the light chain variable region comprises an amino acid sequence selected from the group consisting of SEQ ID NOs:3, 5, and 7.

In some embodiments, the invention provides an anti-Ebola virus envelope glycoprotein antibody, wherein the antibody comprises a heavy chain variable region and a light chain variable region, wherein the heavy chain variable region comprises an amino acid sequence selected from the group consisting of SEQ ID NOs:14, 16, 18, and 20, and wherein the light chain variable region comprises an amino acid sequence selected from the group consisting of SEQ ID NOs:3, 5, and 7.

In some embodiments, the invention provides an anti-Ebola virus envelope glycoprotein antibody, wherein the antibody comprises a heavy chain, wherein the heavy chain comprises an amino acid sequence selected from the group consisting of SEQ ID NOs:15, 17, 19, and 21.

In some embodiments, the invention provides an anti-Ebola virus envelope glycoprotein antibody, wherein the antibody comprises a light chain, wherein the light chain comprises an amino acid sequence selected from the group consisting of SEQ ID NOs:4, 6, and 8.

In some embodiments, the invention provides an anti-Ebola virus envelope glycoprotein antibody, wherein the antibody comprises a heavy chain and a light chain, wherein the heavy chain comprises an amino acid sequence selected from the group consisting of SEQ ID NOs:15, 17, 19, and 21, and wherein the light chain comprises an amino acid sequence selected from the group consisting of SEQ ID NOs:4, 6, and 8.

In some embodiments, the invention provides an anti-Ebola virus envelope glycoprotein antibody: wherein the antibody comprises a heavy chain variable region comprising the amino acid sequence of SEQ ID NO:14, and a light chain variable region comprising the amino acid sequence of SEQ ID NO:3; wherein the antibody comprises a heavy chain variable region comprising the amino acid sequence of SEQ ID NO:16, and a light chain variable region comprising the amino acid sequence of SEQ ID NO:3; wherein the antibody comprises a heavy chain variable region comprising the amino acid sequence of SEQ ID NO:18, and a light chain variable region comprising the amino acid sequence of SEQ ID NO:3;

wherein the antibody comprises a heavy chain variable region comprising the amino acid sequence of SEQ ID NO:20, and a light chain variable region comprising the amino acid sequence of SEQ ID NO:3; wherein the antibody comprises a heavy chain variable region comprising the amino acid sequence of SEQ ID NO:14, and a light chain variable region comprising the amino acid sequence of SEQ ID NO:5; wherein the antibody comprises a heavy chain variable region comprising the amino acid sequence of SEQ ID NO:16, and a light chain variable region comprising the amino acid sequence of SEQ ID NO:5; wherein the antibody comprises a heavy chain variable region comprising the amino acid sequence of SEQ ID NO:18, and a light chain variable region comprising the amino acid sequence of SEQ ID NO:5; wherein the antibody comprises a heavy chain variable region comprising the amino acid sequence of SEQ ID NO:20, and a light chain variable region comprising the amino acid sequence of SEQ ID NO:5; wherein the antibody comprises a heavy chain variable region comprising the amino acid sequence of SEQ ID NO:14, and a light chain variable region comprising the amino acid sequence of SEQ ID NO:7; wherein the antibody comprises a heavy chain variable region comprising the amino acid sequence of SEQ ID NO:16, and a light chain variable region comprising the amino acid sequence of SEQ ID NO:7; wherein the antibody comprises a heavy chain variable region comprising the amino acid sequence of SEQ ID NO:18, and a light chain variable region comprising the amino acid sequence of SEQ ID NO:7; wherein the antibody comprises a heavy chain variable region comprising the amino acid sequence of SEQ ID NO:20, and a light chain variable region comprising the amino acid sequence of SEQ ID NO:7.

In some embodiments, the invention provides an anti-Ebola virus envelope glycoprotein antibody: wherein the antibody comprises a heavy chain comprising the amino acid sequence of SEQ ID NO:15, and a light chain comprising the amino acid sequence of SEQ ID NO:4; wherein the antibody comprises a heavy chain comprising the amino acid sequence of SEQ ID NO:17, and a light chain comprising the amino acid sequence of SEQ ID NO:4; wherein the antibody comprises a heavy chain comprising the amino acid sequence of SEQ ID NO:19, and a light chain comprising the amino acid sequence of SEQ ID NO:4; wherein the antibody comprises a heavy chain comprising the amino acid sequence of SEQ ID NO:21, and a light chain comprising the amino acid sequence of SEQ ID NO:4; wherein the antibody comprises a heavy chain comprising the amino acid sequence of SEQ ID NO:15, and a light chain comprising the amino acid sequence of SEQ ID NO:6; wherein the antibody comprises a heavy chain comprising the amino acid sequence of SEQ ID NO:17, and a light chain comprising the amino acid sequence of SEQ ID NO:6; wherein the antibody comprises a heavy chain comprising the amino acid sequence of SEQ ID NO:19, and a light chain comprising the amino acid sequence of SEQ ID NO:6; wherein the antibody comprises a heavy chain comprising the amino acid sequence of SEQ ID NO:21, and a light chain comprising the amino acid sequence of SEQ ID NO:6; wherein the antibody comprises a heavy chain comprising the amino acid sequence of SEQ ID NO:15, and a light chain comprising the amino acid sequence of SEQ ID NO:8; wherein the antibody comprises a heavy chain comprising the amino acid sequence of SEQ ID NO:17, and a light chain comprising the amino acid sequence of SEQ ID NO:8; wherein the antibody comprises a heavy chain comprising the amino acid sequence of SEQ ID NO:19, and a light chain comprising the amino acid sequence of SEQ ID NO:8; wherein the antibody comprises a heavy chain comprising the amino acid sequence of SEQ ID NO:21, and a light chain comprising the amino acid sequence of SEQ ID NO:8.

In some embodiments, the invention provides an anti-Ebola virus envelope glycoprotein antibody, wherein the antibody comprises (a) HVR-H1 comprising the amino acid sequence of SEQ ID NO:50; (b) HVR-H2 comprising the amino acid sequence of SEQ ID NO:51; and (c) HVR-H3 comprising the amino acid sequence of SEQ ID NO:52.

In some embodiments, the invention provides an anti-Ebola virus envelope glycoprotein antibody, wherein the antibody comprises (a) HVR-L1 comprising the amino acid sequence of SEQ ID NO:37; (b) HVR-L2 comprising the amino acid sequence of SEQ ID NO:38; and (c) HVR-L3 comprising the amino acid sequence of SEQ ID NO:39.

In some embodiments, the invention provides an anti-Ebola virus envelope glycoprotein antibody, wherein the antibody comprises (a) HVR-H1 comprising the amino acid sequence of SEQ ID NO:50; (b) HVR-H2 comprising the amino acid sequence of SEQ ID NO:51; (c) HVR-H3 comprising the amino acid sequence of SEQ ID NO:52; (d) HVR-L1 comprising the amino acid sequence of SEQ ID NO:37; (e) HVR-L2 comprising the amino acid sequence of SEQ ID NO:38; and (f) HVR-L3 comprising the amino acid sequence of SEQ ID NO:39.

In some embodiments, the invention provides an anti-Ebola virus envelope glycoprotein antibody, wherein the antibody comprises a heavy chain variable region, wherein the heavy chain variable region comprises an amino acid sequence selected from the group consisting of SEQ ID NOs:42, 44, 46, and 48.

In some embodiments, the invention provides an anti-Ebola virus envelope glycoprotein antibody, wherein the antibody comprises a light chain variable region, wherein the light chain variable region comprises an amino acid sequence selected from the group consisting of SEQ ID NOs:27, 29, 31, 33, and 35.

In some embodiments, the invention provides an anti-Ebola virus envelope glycoprotein antibody, wherein the antibody comprises a heavy chain variable region and a light chain variable region, wherein the heavy chain variable region comprises an amino acid sequence selected from the group consisting of SEQ ID NOs: 42, 44, 46, and 48, and wherein the light chain variable region comprises an amino acid sequence selected from the group consisting of SEQ ID NOs: 27, 29, 31, 33, and 35.

In some embodiments, the invention provides an anti-Ebola virus envelope glycoprotein antibody, wherein the antibody comprises a heavy chain, wherein the heavy chain comprises an amino acid sequence selected from the group consisting of SEQ ID NOs:43, 45, 47, and 49.

In some embodiments, the invention provides an anti-Ebola virus envelope glycoprotein antibody, wherein the antibody comprises a light chain, wherein the light chain comprises an amino acid sequence selected from the group consisting of SEQ ID NOs:28, 30, 32, 34, and 36.

In some embodiments, the invention provides an anti-Ebola virus envelope glycoprotein antibody, wherein the antibody comprises a heavy chain and a light chain, wherein the heavy chain comprises an amino acid sequence selected from the group consisting of SEQ ID NOs:43, 45, 47, and 49, and wherein the light chain comprises an amino acid sequence selected from the group consisting of SEQ ID NOs:28, 30, 32, 34, and 36.

In some embodiments, the invention provides an anti-Ebola virus envelope glycoprotein antibody: wherein the antibody comprises a heavy chain variable region comprising the amino acid sequence of SEQ ID NO:42, and a light chain variable region comprising the amino acid sequence of SEQ ID NO:27; wherein the antibody comprises a heavy chain variable region comprising the amino acid sequence of SEQ ID NO:44, and a light chain variable region comprising the amino acid sequence of SEQ ID NO:27; wherein the antibody comprises a heavy chain variable region comprising the amino acid sequence of SEQ ID NO:46, and a light chain variable region comprising the amino acid sequence of SEQ ID NO:27; wherein the antibody comprises a heavy chain variable region comprising the amino acid sequence of SEQ ID NO:48, and a light chain variable region comprising the amino acid sequence of SEQ ID NO:27; wherein the antibody comprises a heavy chain variable region comprising the amino acid sequence of SEQ ID NO:42, and a light chain variable region comprising the amino acid sequence of SEQ ID NO:29; wherein the antibody comprises a heavy chain variable region comprising the amino acid sequence of SEQ ID NO:44, and a light chain variable region comprising the amino acid sequence of SEQ ID NO:29; wherein the antibody comprises a heavy chain variable region comprising the amino acid sequence of SEQ ID NO:46, and a light chain variable region comprising the amino acid sequence of SEQ ID NO:29; wherein the antibody comprises a heavy chain variable region comprising the amino acid sequence of SEQ ID NO:48, and a light chain variable region comprising the amino acid sequence of SEQ ID NO:29; wherein the antibody comprises a heavy chain variable region comprising the amino acid sequence of SEQ ID NO:42, and a light chain variable region comprising the amino acid sequence of SEQ ID NO:31; wherein the antibody comprises a heavy chain variable region comprising the amino acid sequence of SEQ ID NO:44, and a light chain variable region comprising the amino acid sequence of SEQ ID NO:31; wherein the antibody comprises a heavy chain variable region comprising the amino acid sequence of SEQ ID NO:46, and a light chain variable region comprising the amino acid sequence of SEQ ID NO:31; wherein the antibody comprises a heavy chain variable region comprising the amino acid sequence of SEQ ID NO:48, and a light chain variable region comprising the amino acid sequence of SEQ ID NO:31; wherein the antibody comprises a heavy chain variable region comprising the amino acid sequence of SEQ ID NO:42, and a light chain variable region comprising the amino acid sequence of SEQ ID NO:33; wherein the antibody comprises a heavy chain variable region comprising the amino acid sequence of SEQ ID NO:44, and a light chain variable region comprising the amino acid sequence of SEQ ID NO:33; wherein the antibody comprises a heavy chain variable region comprising the amino acid sequence of SEQ ID NO:46, and a light chain variable region comprising the amino acid sequence of SEQ ID NO:33; wherein the antibody comprises a heavy chain variable region comprising the amino acid sequence of SEQ ID NO:48, and a light chain variable region comprising the amino acid sequence of SEQ ID NO:33; wherein the antibody comprises a heavy chain variable region comprising the amino acid sequence of SEQ ID NO:42, and a light chain variable region comprising the amino acid sequence of SEQ ID NO:35; wherein the antibody comprises a heavy chain variable region comprising the amino acid sequence of SEQ ID NO:44, and a light chain variable region comprising the amino acid sequence of SEQ ID NO:35; wherein the antibody comprises a heavy chain variable region comprising the amino acid sequence of SEQ ID NO:46, and a light chain variable region comprising the amino acid sequence of SEQ ID NO:35; wherein the antibody comprises a heavy chain variable region comprising the amino acid sequence of SEQ ID NO:48, and a light chain variable region comprising the amino acid sequence of SEQ ID NO:35.

In some embodiments, the invention provides an anti-Ebola virus envelope glycoprotein antibody: wherein the antibody comprises a heavy chain comprising the amino acid sequence of SEQ ID NO:43, and a light chain comprising the amino acid sequence of SEQ ID NO:28; wherein the antibody comprises a heavy chain comprising the amino acid sequence of SEQ ID NO:45, and a light chain comprising the amino acid sequence of SEQ ID NO:28; wherein the antibody comprises a heavy chain comprising the amino acid sequence of SEQ ID NO:47, and a light chain comprising the amino acid sequence of SEQ ID NO:28; wherein the antibody comprises a heavy chain comprising the amino acid sequence of SEQ ID NO:49, and a light chain comprising the amino acid sequence of SEQ ID NO:28; wherein the antibody comprises a heavy chain comprising the amino acid sequence of SEQ ID NO:43, and a light chain comprising the amino acid sequence of SEQ ID NO:30; wherein the antibody comprises a heavy chain comprising the amino acid sequence of SEQ ID NO:45, and a light chain comprising the amino acid sequence of SEQ ID NO:30; wherein the antibody comprises a heavy chain comprising the amino acid sequence of SEQ ID NO:47, and a light chain comprising the amino acid sequence of SEQ ID NO:30; wherein the antibody comprises a heavy chain comprising the amino acid sequence of SEQ ID NO:49, and a light chain comprising the amino acid sequence of SEQ ID NO:30; wherein the antibody comprises a heavy chain comprising the amino acid sequence of SEQ ID NO:43, and a light chain comprising the amino acid sequence of SEQ ID NO:32; wherein the antibody comprises a heavy chain comprising the amino acid sequence of SEQ ID NO:45, and a light chain comprising the amino acid sequence of SEQ ID NO:32; wherein the antibody comprises a heavy chain comprising the amino acid sequence of SEQ ID NO:47, and a light chain comprising the amino acid sequence of SEQ ID NO:32; wherein the antibody comprises a heavy chain comprising the amino acid sequence of SEQ ID NO:49, and a light chain comprising the amino acid sequence of SEQ ID NO:32; wherein the antibody comprises a heavy chain comprising the amino acid sequence of SEQ ID NO:43, and a light chain comprising the amino acid sequence of SEQ ID NO:34; wherein the antibody comprises a heavy chain comprising the amino acid sequence of SEQ ID NO:45, and a light chain comprising the amino acid sequence of SEQ ID NO:34; wherein the antibody comprises a heavy chain comprising the amino acid sequence of SEQ ID NO:47, and a light chain comprising the amino acid sequence of SEQ ID NO:34; wherein the antibody comprises a heavy chain comprising the amino acid sequence of SEQ ID NO:49, and a light chain comprising the amino acid sequence of SEQ ID NO:34; wherein the antibody comprises a heavy chain comprising the amino acid sequence of SEQ ID NO:43, and a light chain comprising the amino acid sequence of SEQ ID NO:36; wherein the antibody comprises a heavy chain comprising the amino acid sequence of SEQ ID NO:45, and a light chain comprising the amino acid sequence of SEQ ID NO:36; wherein the antibody comprises a heavy chain comprising the amino acid sequence of SEQ ID NO:47, and a light chain comprising the amino acid sequence of SEQ ID NO:36; wherein the antibody comprises a heavy chain comprising the amino acid sequence of SEQ ID NO:49, and a light chain comprising the amino acid sequence of SEQ ID NO:36;

In some embodiments, the invention provides an anti-Ebola virus envelope glycoprotein antibody, wherein the antibody comprises (a) HVR-H1 comprising the amino acid sequence of SEQ ID NO:76; (b) HVR-H2 comprising the amino acid sequence of SEQ ID NO:77; and (c) HVR-H3 comprising the amino acid sequence of SEQ ID NO:78.

In some embodiments, the invention provides an anti-Ebola virus envelope glycoprotein antibody, wherein the antibody comprises (a) HVR-L1 comprising the amino acid sequence of SEQ ID NO:65; (b) HVR-L2 comprising the amino acid sequence of SEQ ID NO:66; and (c) HVR-L3 comprising the amino acid sequence of SEQ ID NO:67.

In some embodiments, the invention provides an anti-Ebola virus envelope glycoprotein antibody, wherein the antibody comprises (a) HVR-H1 comprising the amino acid sequence of SEQ ID NO:76; (b) HVR-H2 comprising the amino acid sequence of SEQ ID NO:77; (c) HVR-H3 comprising the amino acid sequence of SEQ ID NO:78; (d) HVR-L1 comprising the amino acid sequence of SEQ ID NO:65; (e) HVR-L2 comprising the amino acid sequence of SEQ ID NO:66; and (f) HVR-L3 comprising the amino acid sequence of SEQ ID NO:67.

In some embodiments, the invention provides an anti-Ebola virus envelope glycoprotein antibody, wherein the antibody comprises a heavy chain variable region, wherein the heavy chain variable region comprises an amino acid sequence selected from the group consisting of SEQ ID NOs:70, 72, and 74.

In some embodiments, the invention provides an anti-Ebola virus envelope glycoprotein antibody, wherein the antibody comprises a light chain variable region, wherein the light chain variable region comprises an amino acid sequence selected from the group consisting of SEQ ID NOs:55, 57, 59, 61, and 63.

In some embodiments, the invention provides an anti-Ebola virus envelope glycoprotein antibody, wherein the antibody comprises a heavy chain variable region and a light chain variable region, wherein the heavy chain variable region comprises an amino acid sequence selected from the group consisting of SEQ ID NOs:70, 72, and 74, and wherein the light chain variable region comprises an amino acid sequence selected from the group consisting of SEQ ID NOs:55, 57, 59, 61, and 63.

In some embodiments, the invention provides an anti-Ebola virus envelope glycoprotein antibody, wherein the antibody comprises a heavy chain, wherein the heavy chain comprises an amino acid sequence selected from the group consisting of SEQ ID NOs:71, 73, and 75.

In some embodiments, the invention provides an anti-Ebola virus envelope glycoprotein antibody, wherein the antibody comprises a light chain, wherein the light chain comprises an amino acid sequence selected from the group consisting of SEQ ID NOs:56, 58, 60, 62, and 64.

In some embodiments, the invention provides an anti-Ebola virus envelope glycoprotein antibody, wherein the antibody comprises a heavy chain and a light chain, wherein the heavy chain comprises an amino acid sequence selected from the group consisting of SEQ ID NOs: 71, 73, and 75, and wherein the light chain comprises an amino acid sequence selected from the group consisting of SEQ ID NOs:56, 58, 60, 62, and 64.

In some embodiments, the invention provides an anti-Ebola virus envelope glycoprotein antibody: wherein the antibody comprises a heavy chain variable region comprising the amino acid sequence of SEQ ID NO:70, and a light chain variable region comprising the amino acid sequence of SEQ ID NO:55; wherein the antibody comprises a heavy chain variable region comprising the amino acid sequence of SEQ ID NO:72, and a light chain variable region comprising the amino acid sequence of SEQ ID NO:55; wherein the antibody comprises a heavy chain variable region comprising the amino acid sequence of SEQ ID NO:74, and a light chain variable region comprising the amino acid sequence of SEQ ID NO:55; wherein the antibody comprises a heavy chain variable region comprising the amino acid sequence of SEQ ID NO:70, and a light chain variable region comprising the amino acid sequence of SEQ ID NO:57; wherein the antibody comprises a heavy chain variable region comprising the amino acid sequence of SEQ ID NO:72, and a light chain variable region comprising the amino acid sequence of SEQ ID NO:57; wherein the antibody comprises a heavy chain variable region comprising the amino acid sequence of SEQ ID NO:74, and a light chain variable region comprising the amino acid sequence of SEQ ID NO:57; wherein the antibody comprises a heavy chain variable region comprising the amino acid sequence of SEQ ID NO:70, and a light chain variable region comprising the amino acid sequence of SEQ ID NO:59; wherein the antibody comprises a heavy chain variable region comprising the amino acid sequence of SEQ ID NO:72, and a light chain variable region comprising the amino acid sequence of SEQ ID NO:59; wherein the antibody comprises a heavy chain variable region comprising the amino acid sequence of SEQ ID NO:74, and a light chain variable region comprising the amino acid sequence of SEQ ID NO:59; wherein the antibody comprises a heavy chain variable region comprising the amino acid sequence of SEQ ID NO:70, and a light chain variable region comprising the amino acid sequence of SEQ ID NO:61; wherein the antibody comprises a heavy chain variable region comprising the amino acid sequence of SEQ ID NO:72, and a light chain variable region comprising the amino acid sequence of SEQ ID NO:61; wherein the antibody comprises a heavy chain variable region comprising the amino acid sequence of SEQ ID NO:74, and a light chain variable region comprising the amino acid sequence of SEQ ID NO:61; wherein the antibody comprises a heavy chain variable region comprising the amino acid sequence of SEQ ID NO:70, and a light chain variable region comprising the amino acid sequence of SEQ ID NO:63; wherein the antibody comprises a heavy chain variable region comprising the amino acid sequence of SEQ ID NO:72, and a light chain variable region comprising the amino acid sequence of SEQ ID NO:63; wherein the antibody comprises a heavy chain variable region comprising the amino acid sequence of SEQ ID NO:74, and a light chain variable region comprising the amino acid sequence of SEQ ID NO:63.

In some embodiments, the invention provides an anti-Ebola virus envelope glycoprotein antibody: wherein the antibody comprises a heavy chain comprising the amino acid sequence of SEQ ID NO:71, and a light chain comprising the amino acid sequence of SEQ ID NO:56; wherein the antibody comprises a heavy chain comprising the amino acid sequence of SEQ ID NO:73, and a light chain comprising the amino acid sequence of SEQ ID NO:56; wherein the antibody comprises a heavy chain comprising the amino acid sequence of SEQ ID NO:75, and a light chain comprising the amino acid sequence of SEQ ID NO:56; wherein the antibody comprises a heavy chain comprising the amino acid sequence of SEQ ID NO:71, and a light chain comprising the amino acid sequence of SEQ ID NO:58; wherein the antibody comprises a heavy chain comprising the amino acid sequence of SEQ ID NO:73, and a light chain comprising the amino acid sequence of SEQ ID NO:58; wherein the antibody comprises a heavy chain comprising the amino acid sequence of SEQ ID NO:75, and a light chain comprising the amino acid sequence of SEQ ID NO:58; wherein the antibody comprises a heavy chain comprising the amino acid sequence of SEQ ID NO:71, and a light chain comprising the amino acid sequence of SEQ ID NO:60; wherein the antibody comprises a heavy chain comprising the amino acid sequence of SEQ ID NO:73, and a light chain comprising the amino acid sequence of SEQ ID NO:60; wherein the antibody comprises a heavy chain comprising the amino acid sequence of SEQ ID NO:75, and a light chain comprising the amino acid sequence of SEQ ID NO:60; wherein the antibody comprises a heavy chain comprising the amino acid sequence of SEQ ID NO:71, and a light chain comprising the amino acid sequence of SEQ ID NO:62; wherein the antibody comprises a heavy chain comprising the amino acid sequence of SEQ ID NO:73, and a light chain comprising the amino acid sequence of SEQ ID NO:62; wherein the antibody comprises a heavy chain comprising the amino acid sequence of SEQ ID NO:75, and a light chain comprising the amino acid sequence of SEQ ID NO:62; wherein the antibody comprises a heavy chain comprising the amino acid sequence of SEQ ID NO:71, and a light chain comprising the amino acid sequence of SEQ ID NO:64; wherein the antibody comprises a heavy chain comprising the amino acid sequence of SEQ ID NO:73, and a light chain comprising the amino acid sequence of SEQ ID NO:64; wherein the antibody comprises a heavy chain comprising the amino acid sequence of SEQ ID NO:75, and a light chain comprising the amino acid sequence of SEQ ID NO:64.

In some embodiments, the invention provides an anti-Ebola virus envelope glycoprotein antibody, wherein the antibody comprises (a) a heavy chain variable region amino acid sequence having at least 90% amino acid sequence identity to the amino acid sequence of SEQ ID NO:14, (b) a light chain variable region amino acid sequence having at least 90% amino acid sequence identity to the amino acid sequence of SEQ ID NO:7, or (c) a heavy chain variable region amino acid sequence region as in (a) and a light chain variable region amino acid sequence as in (b).

In some embodiments, the invention provides an anti-Ebola virus envelope glycoprotein antibody, wherein the antibody comprises (a) a heavy chain variable region amino acid sequence having at least 90% amino acid sequence identity to the amino acid sequence of SEQ ID NO:42, (b) a light chain variable region amino acid sequence having at least 90% amino acid sequence identity to the amino acid sequence of SEQ ID NO:35, or (c) a heavy chain variable region amino acid sequence region as in (a) and a light chain variable region amino acid sequence as in (b).

In some embodiments, the invention provides an anti-Ebola virus envelope glycoprotein antibody, wherein the antibody comprises (a) a heavy chain variable region amino acid sequence having at least 90% amino acid sequence identity to the amino acid sequence of SEQ ID NO:70, (b) a light chain variable region amino acid sequence having at least 90% amino acid sequence identity to the amino acid sequence of SEQ ID NO:55, or (c) a heavy chain variable region amino acid sequence region as in (a) and a light chain variable region amino acid sequence as in (b).

In some embodiments, the invention provides an anti-Ebola virus envelope glycoprotein antibody, wherein the antibody binds to an Ebola virus glycoprotein comprising a sequence selected from the group consisting of SEQ ID NOS:79-90.

In certain embodiments, an anti-Ebola virus envelope glycoprotein antibody of the present invention is an antibody fragment. In other embodiments, an anti-Ebola virus envelope glycoprotein antibody of the present invention is a full length IgG1 antibody. In other embodiments, an anti-Ebola virus envelope glycoprotein antibody of the present invention is afucosylated.

The present invention further provides an isolated nucleic acid molecule encoding an anti-Ebola virus envelope glycoprotein antibody of the present invention. The invention also provides vectors comprising a nucleic acid molecule encoding an anti-Ebola virus envelop glycoprotein of the present invention. The vector can be of any type, for example, a recombinant vector such as an expression vector.

In some aspects, the invention provides a host cell comprising a nucleic acid molecule encoding an anti-Ebola virus envelope glycoprotein antibody of the present invention. Any of a variety of host cells can be used. In one embodiment, a host cell is a prokaryotic cell, for example, E. coli. In another embodiment, the host cell is a eukaryotic cell, for example, a mammalian cell, such as a Chinese Hampster Ovary (CHO) cell, or a plant cell, such as a tobacco cell. In some embodiments, the host cell is a host cell that is capable of producing afucosylated antibodies. In other embodiments, the host cell is a mammalian host cell that is capable of producing afucosylated antibodies. In other embodiments, the host cell is a mammalian host cell that has a deletion in the FUT8 (alpha-(1,6)-fucosyltransferase) gene which results in no fucose addition to expressed proteins.

The invention further provides a method of producing an anti-Ebola virus envelope glycoprotein antibody of the present invention. For example, the invention provides methods for making an anti-Ebola virus envelope glycoprotein antibody of the present invention (which, as defined herein, includes full length antibody and fragments thereof), the method comprising expressing in a suitable host cell a recombinant vector of the invention encoding the anti-Ebola virus envelope glycoprotein antibody of the invention or fragments thereof so that the antibody or fragments thereof are produced. In some embodiments, the method comprises culturing a host cell comprising nucleic acid encoding an anti-Ebola virus envelope glycoprotein antibody of the present invention (or fragments thereof) so that the nucleic acid is expressed. The method may further comprise recovering the anti-Ebola virus envelope glycoprotein antibody or fragments thereof from the host cell, from the host cell culture, or from the host cell culture medium. In some embodiments, the method described herein results in decreased antibody aggregation, relative to producing the antibody in a host cell that does not produce afucosylated antibodies.

In some embodiments, the invention provides an immunoconjugate comprising an anti-Ebola virus envelope glycoprotein antibody of the present invention and a cytotoxic agent.

In some embodiments, the invention provides a pharmaceutical formulation comprising an anti-Ebola virus envelope glycoprotein antibody of the present invention. In some embodiments, the invention provides a pharmaceutical formulation comprising an anti-Ebola virus envelope glycoprotein antibody of the present invention and/or an immunoconjugate and a pharmaceutically acceptable carrier or diluent. In some embodiments, the pharmaceutical formulation further comprises an additional therapeutic agent, such as another anti-Ebola virus antibody, an anti-viral agent, or an anti-Ebola vaccine. In some embodiments, the invention provides a pharmaceutical composition comprising an anti-Ebola virus envelope glycoprotein antibody of the present invention for use in treating, preventing, or inhibiting Ebola virus infection.

In some embodiments, the invention provides an anti-Ebola virus envelope glycoprotein antibody of the present invention for use as a medicament. In some embodiments, the invention provides an anti-Ebola virus envelope glycoprotein antibody of the present invention for use in treating Ebola virus infection. In some embodiments, the invention provides for use of an anti-Ebola virus envelope glycoprotein antibody of the present invention in the manufacture of a medicament for treating Ebola virus infection. In another aspect, the invention provides for use of an anti-Ebola virus envelope glycoprotein antibody of the present invention in the manufacture of a medicament. The medicament may be for use in the inhibition, treatment, or prevention of Ebola virus infection. In another aspect, the invention provides for use of an article of manufacture of the invention in the manufacture of a medicament. The medicament may be for use in the inhibition, treatment, or prevention of Ebola virus infection.

In some embodiments, the present invention provides a method of treating an individual having Ebola virus infection comprising administering to the individual an effective amount of an anti-Ebola virus envelope glycoprotein antibody of the present invention or an immunoconjugate thereof. In some embodiments, the method further comprises administering an additional therapeutic agent to the individual. In some embodiments, the additional therapeutic agent is another anti-Ebola virus antibody, an anti-viral agent, or an anti-Ebola vaccine.

The invention also provides a method for inhibiting Ebola virus infection, the method comprising administering to an individual in need thereof an effective amount of a composition comprising one or more of the anti-Ebola virus envelope glycoprotein antibodies of the present invention, thereby inhibiting Ebola virus infection. The invention also provides a method for treating Ebola virus infection, the method comprising administering to an individual in need thereof an effective amount of a composition comprising one or more of the anti-Ebola virus envelope glycoprotein antibodies of the present invention, thereby treating Ebola virus infection. The invention also provides a method for preventing Ebola virus infection, the method comprising administering to an individual in need thereof an effective amount of a composition comprising one or more of the anti-Ebola virus envelope glycoprotein antibodies of the present invention, thereby preventing Ebola virus infection.

In some embodiments, an anti-Ebola virus envelope glycoprotein antibody of the present invention neutralizes Ebola virus in vitro, in vivo, or in vitro and in vivo. In some embodiments, an anti-Ebola virus envelope glycoprotein antibody of the present invention is a monoclonal antibody. In some embodiments, an anti-Ebola virus envelope glycoprotein antibody of the present invention is an isolated monoclonal antibody.

### BRIEF DESCRIPTION OF THE FIGURES

FIG. 1A sets forth the amino acid sequences of light chain variable region (SEQ ID NO:1) and light chain (SEQ ID NO:2) of chimeric anti-Ebola virus monoclonal antibody 13C6. The hypervariable regions are underlined.

FIG. 1B sets forth the amino acid sequences of light chain variable region (SEQ ID NO:3) and light chain (SEQ ID NO:4) of humanized anti-Ebola virus monoclonal antibody 13C6 graft to kappa1. Underlined amino acid residues indicate amino acid positions in the variable region which differ between the chimeric and humanized anti-Ebola virus monoclonal antibody 13C6.

FIG. 1C sets forth the amino acid sequences of light chain variable region (SEQ ID NO:5) and light chain (SEQ ID NO:6) of humanized anti-Ebola virus monoclonal antibody 13C6 graft to kappa1 with murine substitution at Vernier postion 43 (Kabat numbering). Underlined amino acid residues indicate amino acid positions in the variable region which differ between the chimeric and humanized anti-Ebola virus monoclonal antibody 13C6.

FIG. 1D sets forth the amino acid sequences of light chain variable region (SEQ ID NO:7) and light chain (SEQ ID NO:8) of humanized anti-Ebola virus monoclonal antibody 13C6 graft to kappa1 with murine substitution at Vernier postions 43 and 87 (Kabat numbering). Underlined amino acid residues indicate amino acid positions in the variable region which differ between the chimeric and humanized anti-Ebola virus monoclonal antibody 13C6.

FIG. 1E sets forth the amino acid sequence of the light chain hypervariable region (HVR-L1; SEQ ID NO:9) of the chimeric and humanized anti-Ebola virus monoclonal antibody 13C6.

FIG. 1F sets forth the amino acid sequence of the light chain hypervariable region (HVR-L2; SEQ ID NO:10) of the chimeric and humanized anti-Ebola virus monoclonal antibody 13C6.

FIG. 1G sets forth the amino acid sequence of the light chain hypervariable region (HVR-L3; SEQ ID NO:11) of the chimeric and humanized anti-Ebola virus monoclonal antibody 13C6.

FIG. 1H sets forth the amino acid sequences of heavy chain variable region (SEQ ID NO:12) and heavy chain (SEQ ID NO:13) of chimeric anti-Ebola virus monoclonal antibody 13C6. The hypervariable regions are underlined.

FIG. 1I sets forth the amino acid sequences of heavy chain variable region (SEQ ID NO:14) and heavy chain (SEQ ID NO:15) of humanized anti-Ebola virus monoclonal antibody 13C6 graft to H2 germline. Underlined amino acid residues indicate amino acid positions in the variable region which differ between the chimeric and humanized anti-Ebola virus monoclonal antibody 13C6.

FIG. 1J sets forth the amino acid sequences of heavy chain variable region (SEQ ID NO:16) and heavy chain (SEQ ID NO:17) of humanized anti-Ebola virus monoclonal antibody 13C6 graft to H2 germline with murine substitution at Vernier positions 2, 24, and 37 (Kabat numbering). Underlined amino acid residues indicate amino acid positions in the variable region which differ between the chimeric and humanized anti-Ebola virus monoclonal antibody 13C6.

FIG. 1K sets forth the amino acid sequences of heavy chain variable region (SEQ ID NO:18) and heavy chain (SEQ ID NO:19) of humanized anti-Ebola virus monoclonal antibody 13C6 graft to H2 germline with murine substitution at Vernier positions 2, 24, 37, 73, and 75 (Kabat numbering). Underlined amino acid residues indicate amino acid positions in the variable region which differ between the chimeric and humanized anti-Ebola virus monoclonal antibody 13C6.

FIG. 1L sets forth the amino acid sequences of heavy chain variable region (SEQ ID NO:20) and heavy chain (SEQ ID NO:21) of humanized anti-Ebola virus monoclonal antibody 13C6 graft to H2 germline with murine substitution at Vernier positions 2, 24, 37, 66, 68, 73, and 75 (Kabat numbering). Underlined amino acid residues indicate amino acid positions in the variable region which differ between the chimeric and humanized anti-Ebola virus monoclonal antibody 13C6.

FIG. 1M sets forth the amino acid sequence of the heavy chain hypervariable region (HVR-H1; SEQ ID NO:22) of the chimeric and humanized anti-Ebola virus monoclonal antibody 13C6.

FIG. 1N sets forth the amino acid sequence of the heavy chain hypervariable region (HVR-H2; SEQ ID NO:23) of the chimeric and humanized anti-Ebola virus monoclonal antibody 13C6.

FIG. 1O sets forth the amino acid sequence of the heavy chain hypervariable region (HVR-H3; SEQ ID NO:24) of the chimeric and humanized anti-Ebola virus monoclonal antibody 13C6.

FIG. 2A sets forth the amino acid sequences of light chain variable region (SEQ ID NO:25) and light chain (SEQ ID NO:26) of chimeric anti-Ebola virus monoclonal antibody 2G4. The hypervariable regions are underlined.

FIG. 2B sets forth the amino acid sequences of light chain variable region (SEQ ID NO:27) and light chain (SEQ ID NO:28) of humanized anti-Ebola virus monoclonal antibody 2G4 graft to kappa1. Underlined amino acid residues indicate amino acid positions in the variable region which differ between the chimeric and humanized anti-Ebola virus monoclonal antibody 2G4.

FIG. 2C sets forth the amino acid sequences of light chain variable region (SEQ ID NO:29) and light chain (SEQ ID NO:30) of humanized anti-Ebola virus monoclonal antibody 2G4 graft to kappa1 with murine substitution at Vernier position 43 (Kabat numbering). Underlined amino acid residues indicate amino acid positions in the variable region which differ between the chimeric and humanized anti-Ebola virus monoclonal antibody 2G4.

FIG. 2D sets forth the amino acid sequences of light chain variable region (SEQ ID NO:31) and light chain (SEQ ID NO:32) of humanized anti-Ebola virus monoclonal antibody 2G4 graft to kappa1 with murine substitution at Vernier position 48 (Kabat numbering). Underlined amino acid residues indicate amino acid positions in the variable region which differ between the chimeric and humanized anti-Ebola virus monoclonal antibody 2G4.

FIG. 2E sets forth the amino acid sequences of light chain variable region (SEQ ID NO:33) and light chain (SEQ ID NO:34) of humanized anti-Ebola virus monoclonal antibody 2G4 graft to kappa1 with murine substitution at Vernier positions 43 and 48 (Kabat numbering). Underlined amino acid residues indicate amino acid positions in the variable region which differ between the chimeric and humanized anti-Ebola virus monoclonal antibody 2G4.

FIG. 2F sets forth the amino acid sequences of light chain variable region (SEQ ID NO:35) and light chain (SEQ ID NO:36) of humanized anti-Ebola virus monoclonal antibody 2G4 graft to kappa1 with murine substitution at Vernier positions 43, 48, and 71 (Kabat numbering). Underlined amino acid residues indicate amino acid positions in the variable region which differ between the chimeric and humanized anti-Ebola virus monoclonal antibody 2G4.

FIG. 2G sets forth the amino acid sequence of the light chain hypervariable region (HVR-L1; SEQ ID NO:37) of the chimeric and humanized anti-Ebola virus monoclonal antibody 2G4.

FIG. 2H sets forth the amino acid sequence of the light chain hypervariable region (HVR-L2; SEQ ID NO:38) of the chimeric and humanized anti-Ebola virus monoclonal antibody 2G4.

FIG. 2I sets forth the amino acid sequence of the light chain hypervariable region (HVR-L3; SEQ ID NO:39) of the chimeric and humanized anti-Ebola virus monoclonal antibody 2G4.

FIG. 2J sets forth the amino acid sequences of heavy chain variable region (SEQ ID NO:40) and heavy chain (SEQ ID NO:41) of chimeric anti-Ebola virus monoclonal antibody 2G4. The hypervariable regions are underlined.

FIG. 2K sets forth the amino acid sequences of heavy chain variable region (SEQ ID NO:42) and heavy chain (SEQ ID NO:43) of humanized anti-Ebola virus monoclonal antibody 2G4 graft to H3 germline. Underlined amino acid residues indicate amino acid positions in the variable region which differ between the chimeric and humanized anti-Ebola virus monoclonal antibody 2G4.

FIG. 2L sets forth the amino acid sequences of heavy chain variable region (SEQ ID NO:44) and heavy chain (SEQ ID NO:45) of humanized anti-Ebola virus monoclonal antibody 2G4 graft to H3 germline with murine substitution at Vernier position 49 (Kabat numbering). Underlined amino acid residues indicate amino acid positions in the variable region which differ between the chimeric and humanized anti-Ebola virus monoclonal antibody 2G4.

FIG. 2M sets forth the amino acid sequences of heavy chain variable region (SEQ ID NO:46) and heavy chain (SEQ ID NO:47) of humanized anti-Ebola virus monoclonal antibody 2G4 graft to H3 germline with murine substitution at Vernier position 78 (Kabat numbering). Underlined amino acid residues indicate amino acid positions in the variable region which differ between the chimeric and humanized anti-Ebola virus monoclonal antibody 2G4.

FIG. 2N sets forth the amino acid sequences of heavy chain variable region (SEQ ID NO:48) and heavy chain (SEQ ID NO:49) of humanized anti-Ebola virus monoclonal antibody 2G4 graft to H3 germline with murine substitution at Vernier positions 49 and 78 (Kabat numbering). Underlined amino acid residues indicate amino acid positions in the variable region which differ between the chimeric and humanized anti-Ebola virus monoclonal antibody 2G4.

FIG. 2O sets forth the amino acid sequence of the heavy chain hypervariable region (HVR-H1; SEQ ID NO:50) of the chimeric and humanized anti-Ebola virus monoclonal antibody 2G4.

FIG. 2P sets forth the amino acid sequence of the heavy chain hypervariable region (HVR-H1; SEQ ID NO:51) of the chimeric and humanized anti-Ebola virus monoclonal antibody 2G4.

FIG. 2Q sets forth the amino acid sequence of the heavy chain hypervariable region (HVR-H1; SEQ ID NO:52) of the chimeric and humanized anti-Ebola virus monoclonal antibody 2G4.

FIG. 3A sets forth the amino acid sequences of light chain variable region (SEQ ID NO:53) and light chain (SEQ ID NO:54) of chimeric anti-Ebola virus monoclonal antibody 4G7. The hypervariable regions are underlined.

FIG. 3B sets forth the amino acid sequences of light chain variable region (SEQ ID NO:55) and light chain (SEQ ID NO:56) of humanized anti-Ebola virus monoclonal antibody 4G7 graft to kappa1. Underlined amino acid residues indicate amino acid positions in the variable region which differ between the chimeric and humanized anti-Ebola virus monoclonal antibody 4G7.

FIG. 3C sets forth the amino acid sequences of light chain variable region (SEQ ID NO:57) and light chain (SEQ ID NO:58) of humanized anti-Ebola virus monoclonal antibody 4G7 graft to kappa1 with murine substitution at Vernier position 43 (Kabat numbering). Underlined amino acid residues indicate amino acid positions in the variable region which differ between the chimeric and humanized anti-Ebola virus monoclonal antibody 4G7.

FIG. 3D sets forth the amino acid sequences of light chain variable region (SEQ ID NO:59) and light chain (SEQ ID NO:60) of humanized anti-Ebola virus monoclonal antibody 4G7 graft to kappa1 with murine substitution at Vernier position 48 (Kabat numbering). Underlined amino acid residues indicate amino acid positions in the variable region which differ between the chimeric and humanized anti-Ebola virus monoclonal antibody 4G7.

FIG. 3E sets forth the amino acid sequences of light chain variable region (SEQ ID NO:61) and light chain (SEQ ID NO:62) of humanized anti-Ebola virus monoclonal antibody 4G7 graft to kappa1 with murine substitution at Vernier positions 43 and 48 (Kabat numbering). Underlined amino acid residues indicate amino acid positions in the variable region which differ between the chimeric and humanized anti-Ebola virus monoclonal antibody 4G7.

FIG. 3F sets forth the amino acid sequences of light chain variable region (SEQ ID NO:63) and light chain (SEQ ID NO:64) of humanized anti-Ebola virus monoclonal antibody 4G7 graft to kappa1 with murine substitution at Vernier positions 43, 48, and 87 (Kabat numbering). Underlined amino acid residues indicate amino acid positions in the variable region which differ between the chimeric and humanized anti-Ebola virus monoclonal antibody 4G7.

FIG. 3G sets forth the amino acid sequence of the light chain hypervariable region (HVR-L1; SEQ ID NO:65) of the chimeric and humanized anti-Ebola virus monoclonal antibody 4G7.

FIG. 3H sets forth the amino acid sequence of the light chain hypervariable region (HVR-L2; SEQ ID NO:66) of the chimeric and humanized anti-Ebola virus monoclonal antibody 4G7.

FIG. 3I sets forth the amino acid sequence of the light chain hypervariable region (HVR-L3; SEQ ID NO:67) of the chimeric and humanized anti-Ebola virus monoclonal antibody 4G7.

FIG. 3J sets forth the amino acid sequences of heavy chain variable region (SEQ ID NO:68) and heavy chain (SEQ ID NO:69) of chimeric anti-Ebola virus monoclonal antibody 4G7. The hypervariable regions are underlined.

FIG. 3K sets forth the amino acid sequences of heavy chain variable region (SEQ ID NO:70) and heavy chain (SEQ ID NO:71) of humanized anti-Ebola virus monoclonal antibody 4G7 graft to H1 germline. Underlined amino acid residues indicate amino acid positions in the variable region which differ between the chimeric and humanized anti-Ebola virus monoclonal antibody 4G7.

FIG. 3L sets forth the amino acid sequences of heavy chain variable region (SEQ ID NO:72) and heavy chain (SEQ ID NO:73) of humanized anti-Ebola virus monoclonal antibody 4G7 graft to H1 germline with murine substitution at Vernier position 71 (Kabat numbering). Underlined amino acid residues indicate amino acid positions in the variable region which differ between the chimeric and humanized anti-Ebola virus monoclonal antibody 4G7.

FIG. 3M sets forth the amino acid sequences of heavy chain variable region (SEQ ID NO:74) and heavy chain (SEQ ID NO:75) of humanized anti-Ebola virus monoclonal antibody 4G7 graft to H1 germline with murine substitution at Vernier positions 67, 69, and 71 (Kabat numbering). Underlined amino acid residues indicate amino acid positions in the variable region which differ between the chimeric and humanized anti-Ebola virus monoclonal antibody 4G7.

FIG. 3N sets forth the amino acid sequence of the heavy chain hypervariable region (HVR-H1; SEQ ID NO:76) of the chimeric and humanized anti-Ebola virus monoclonal antibody 4G7.

FIG. 3O sets forth the amino acid sequence of the heavy chain hypervariable region (HVR-H2; SEQ ID NO:77) of the chimeric and humanized anti-Ebola virus monoclonal antibody 4G7.

FIG. 3P sets forth the amino acid sequence of the heavy chain hypervariable region (HVR-H3; SEQ ID NO:78) of the chimeric and humanized anti-Ebola virus monoclonal antibody 4G7.

FIG. 4A-FIG. 4L set forth the amino acid sequences (SEQ ID NOS:79-90) of virus envelope glycoprotein from different strains of Ebola virus species.

FIG. 5A-FIG. 5B set forth the epitope sequences of the anti-Ebola virus monoclonal antibodies 13C6 (SEQ ID NO:91), 2G4 (SEQ ID NO:92), and 4G7 (SEQ ID NO:92).

### DETAILED DESCRIPTION OF EMBODIMENTS OF THE INVENTION

### I. Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL or VH acceptor human framework is identical in sequence to the VL or VH human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

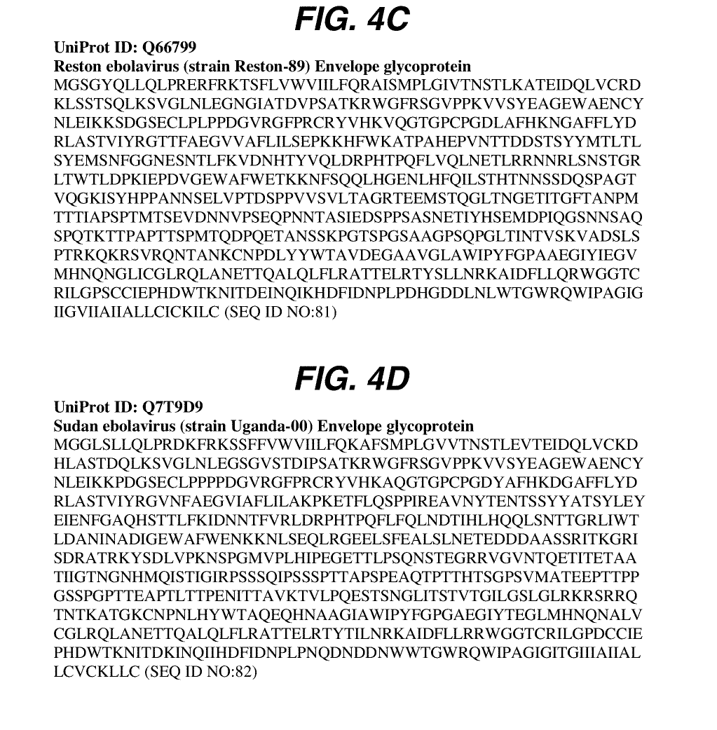

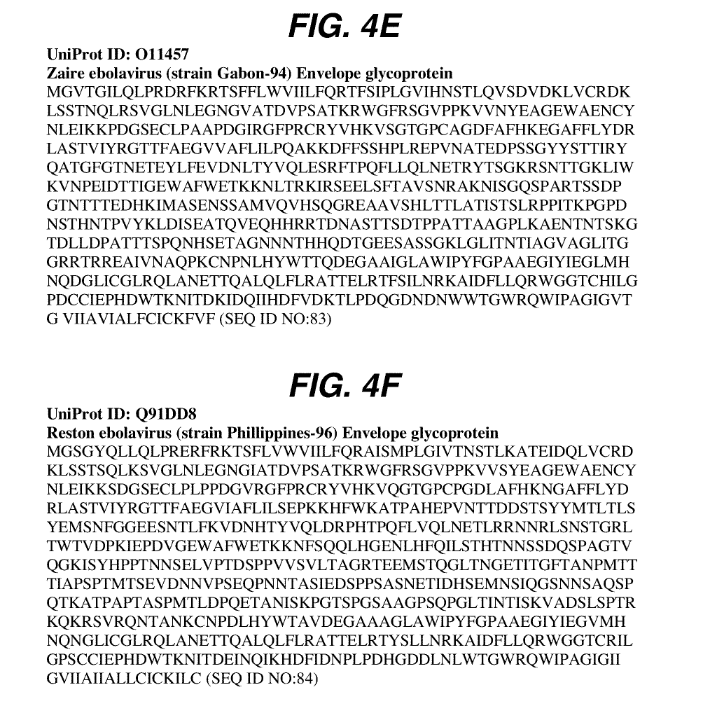

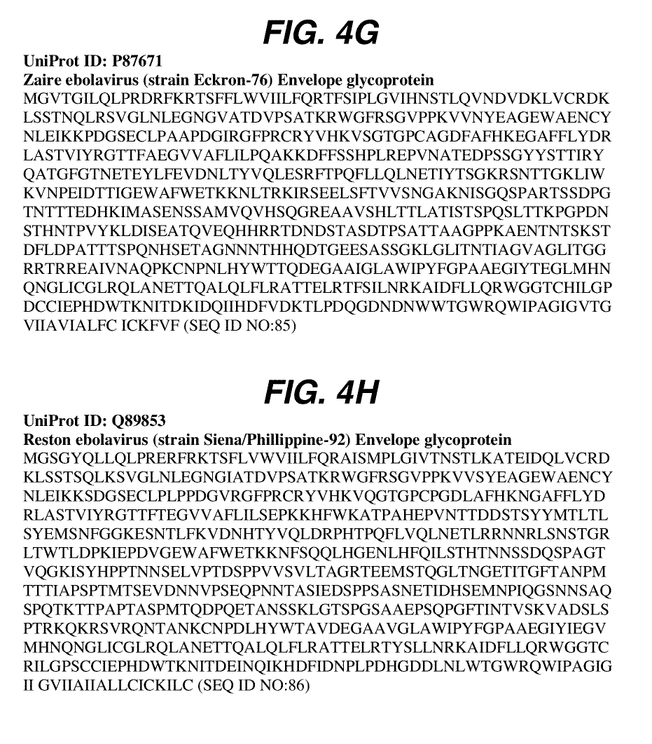

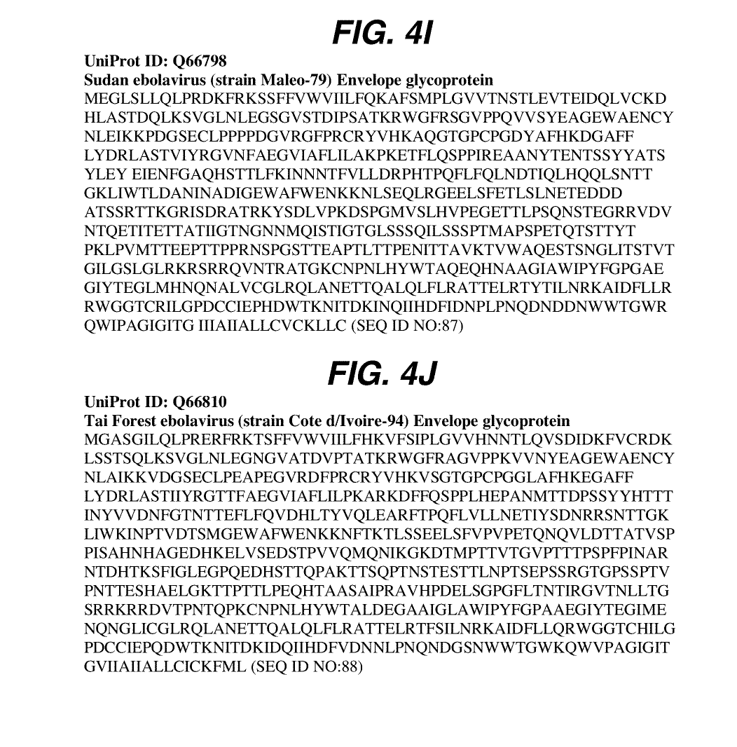

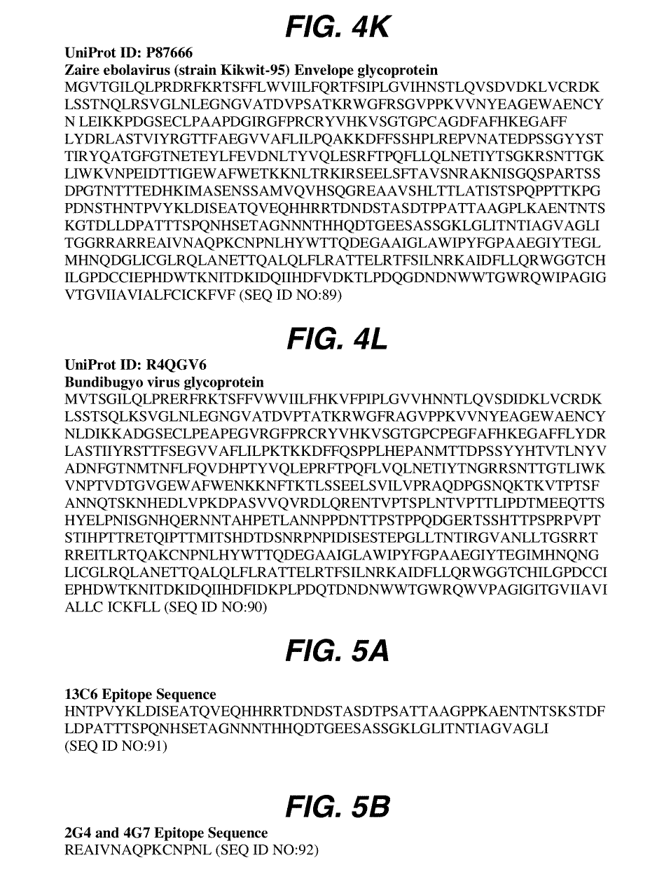

The terms “anti-Ebola virus glycoprotein antibody,” “anti-Ebola virus envelop glycoprotein antibody,” “an antibody that binds to Ebola virus glycoprotein,” “anti-Ebola virus GP antibody,” and “an antibody that binds to Ebola virus GP” refer to an antibody that is capable of binding Ebola virus envelop glycoprotein with sufficient affinity such that the antibody is useful as a diagnostic and/or therapeutic agent in targeting Ebola virus glycoprotein. In one embodiment, the extent of binding of an anti-Ebola virus glycoprotein antibody to an unrelated, non-Ebola virus glycoprotein protein is less than about 10% of the binding of the antibody to Ebola virus glycoprotein as measured, e.g., by a radioimmunoassay (RIA). In certain embodiments, an antibody that binds to Ebola virus glycoprotein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸M or less, e.g. from 10⁻⁸M to 10⁻¹³M, e.g., from 10⁻⁹M to 10⁻¹³ M). In certain embodiments, an anti-Ebola virus glycoprotein antibody binds to an epitope of Ebola virus glycoprotein that is conserved among Ebola virus glycoprotein from different Ebola virus species within the Ebola virus genus (e.g., Zaire ebolavirus envelope glycoprotein, Sudan ebolavirus envelope glycoprotein, Reston ebolavirus envelope glycoprotein, Tai Forest ebolavirus envelope glycoprotein, Bundibugyo ebolavirus envelope glycoprotein; see, e.g., FIG. 4A-FIG. 4L, SEQ ID NOS:79-90).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

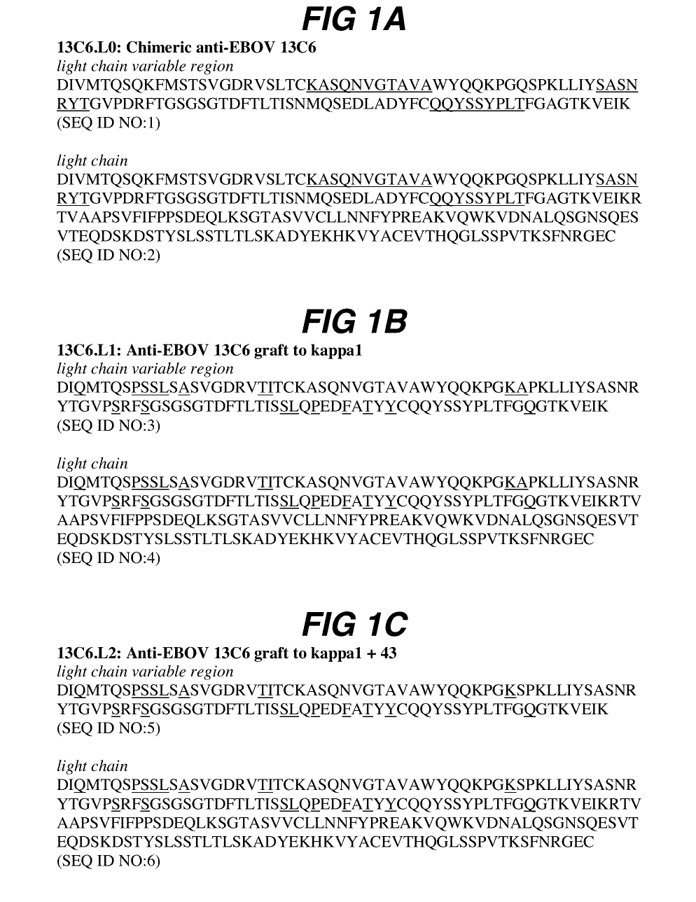

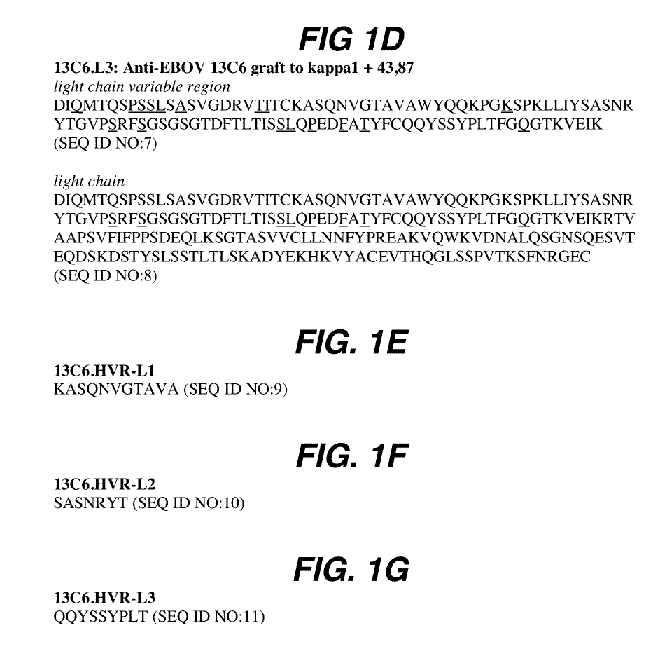

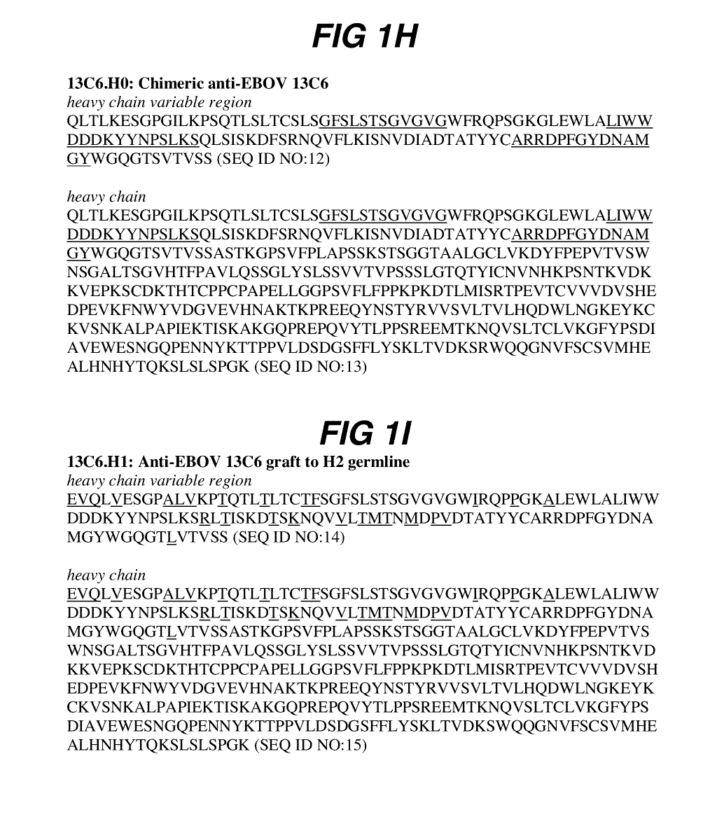

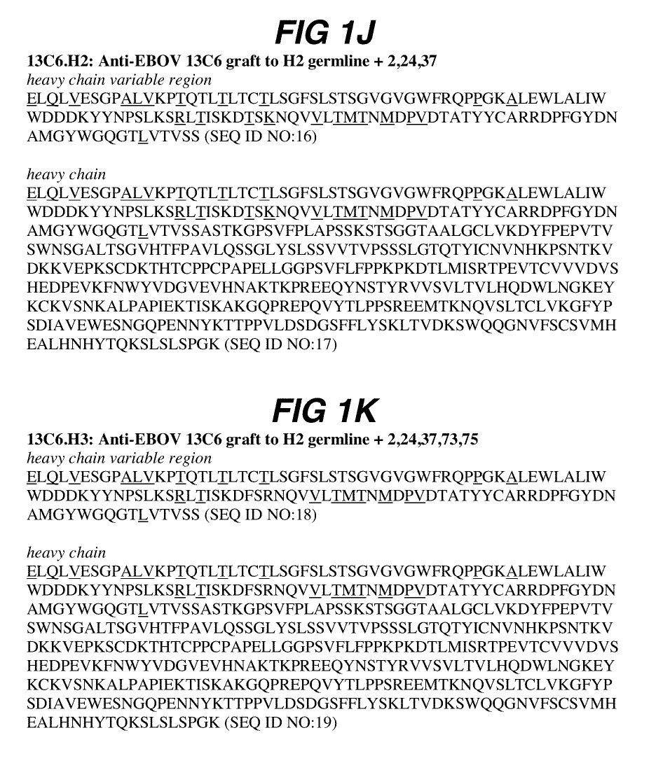

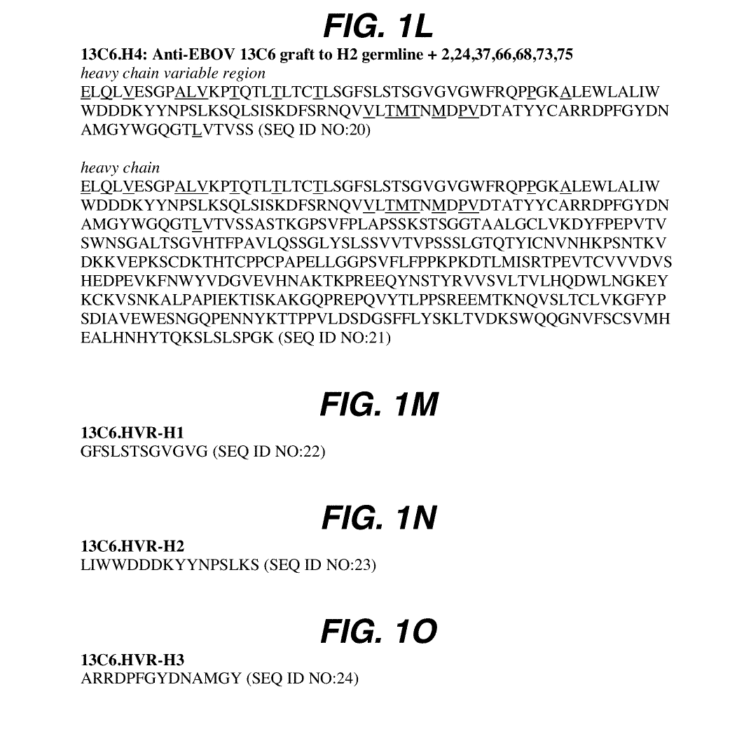

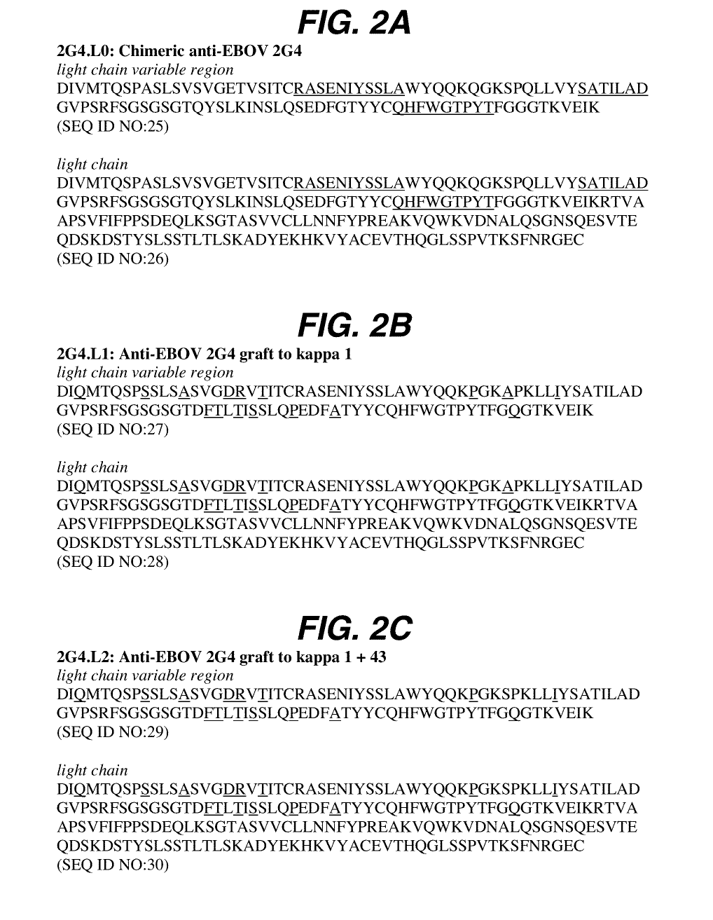

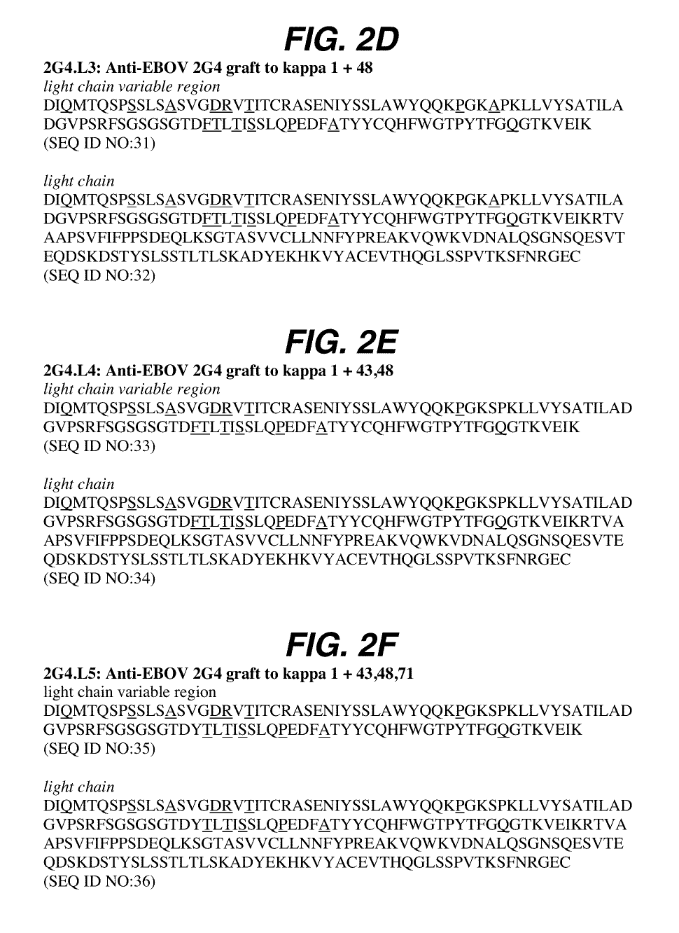

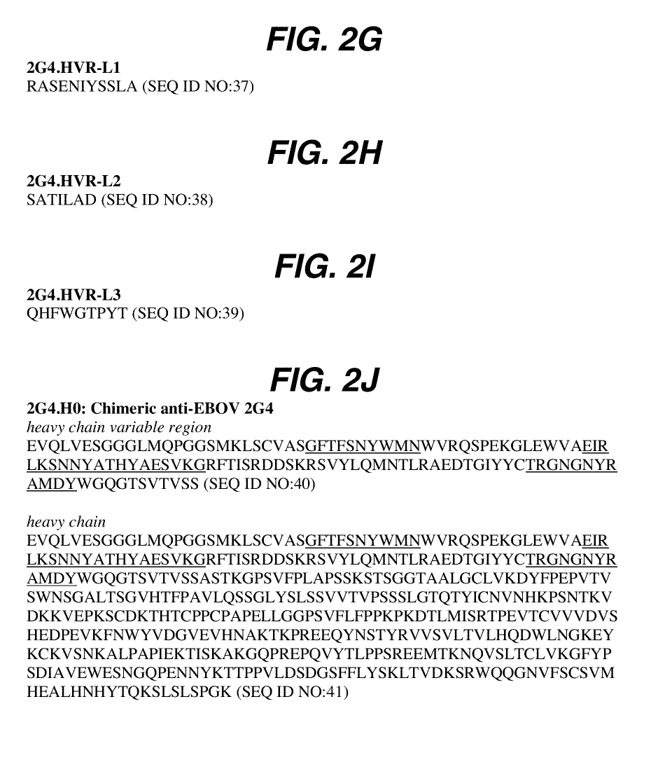

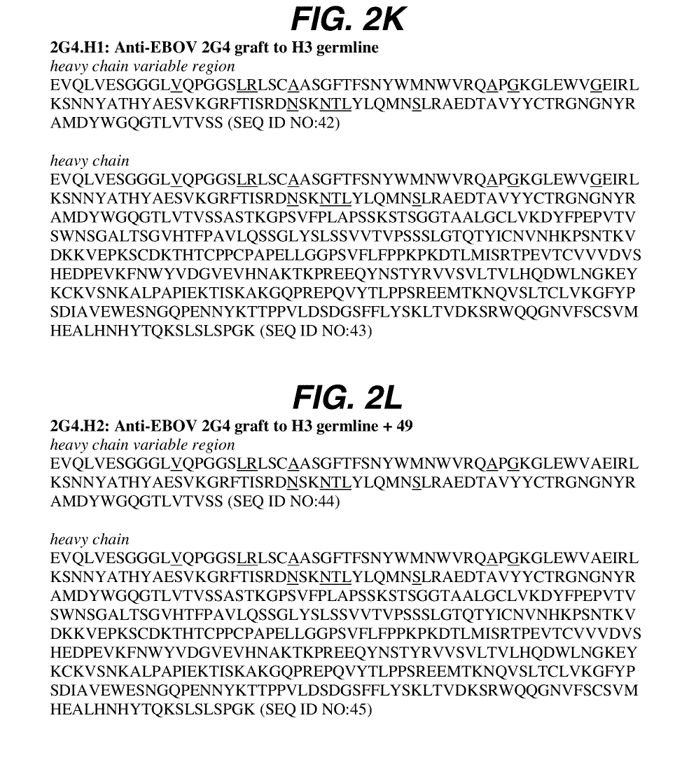

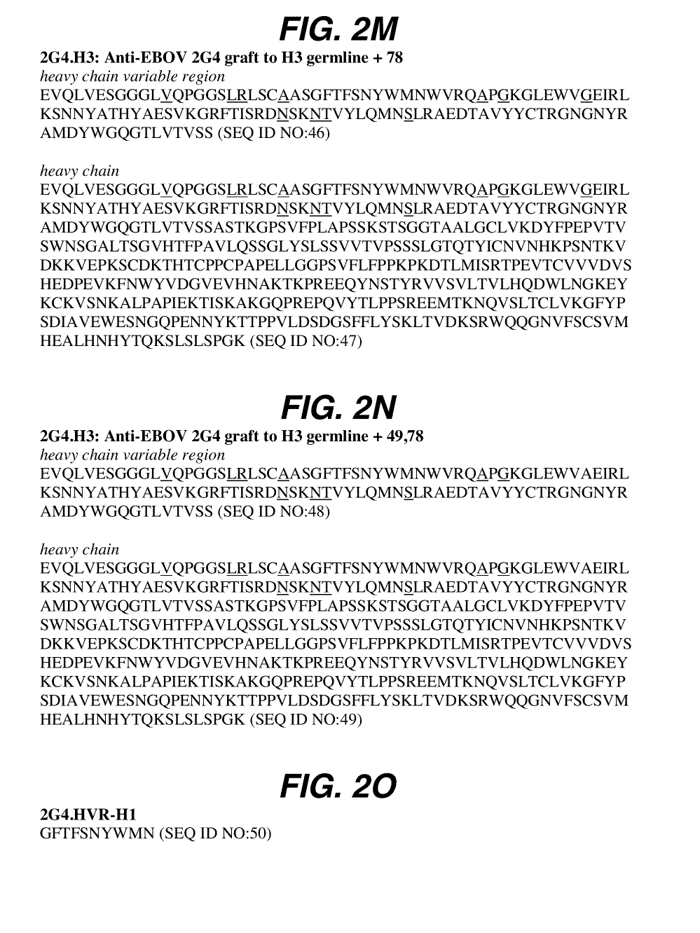

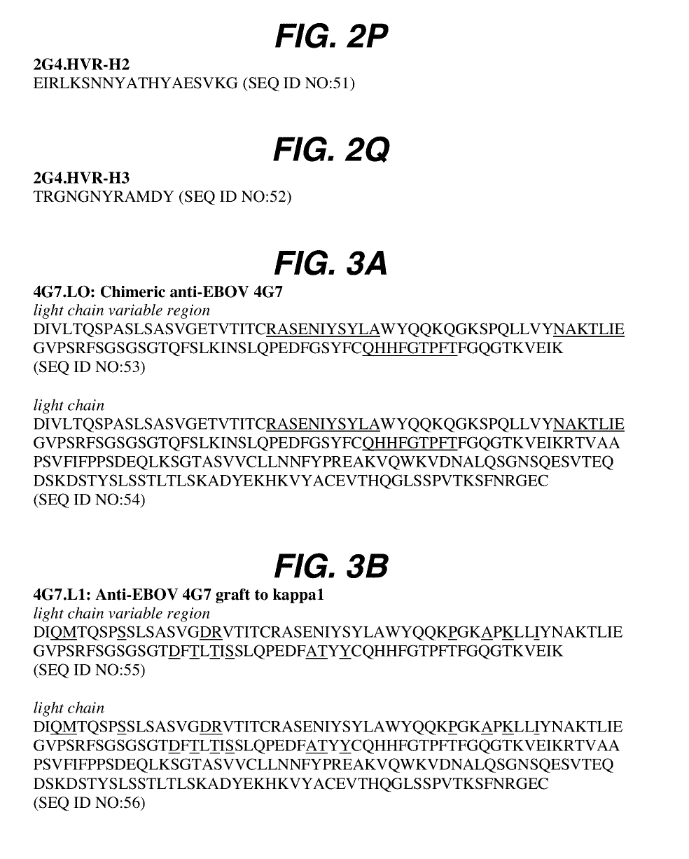

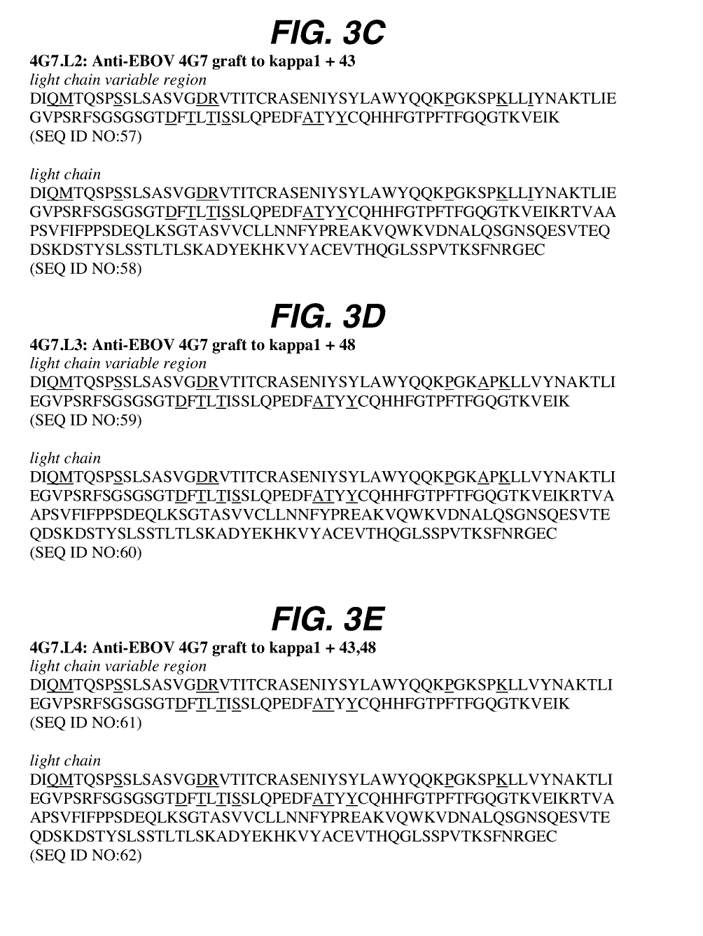

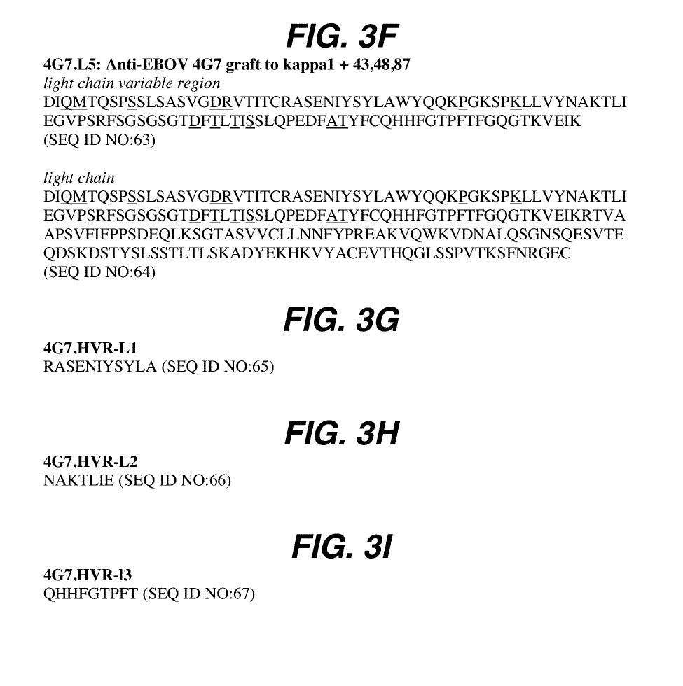

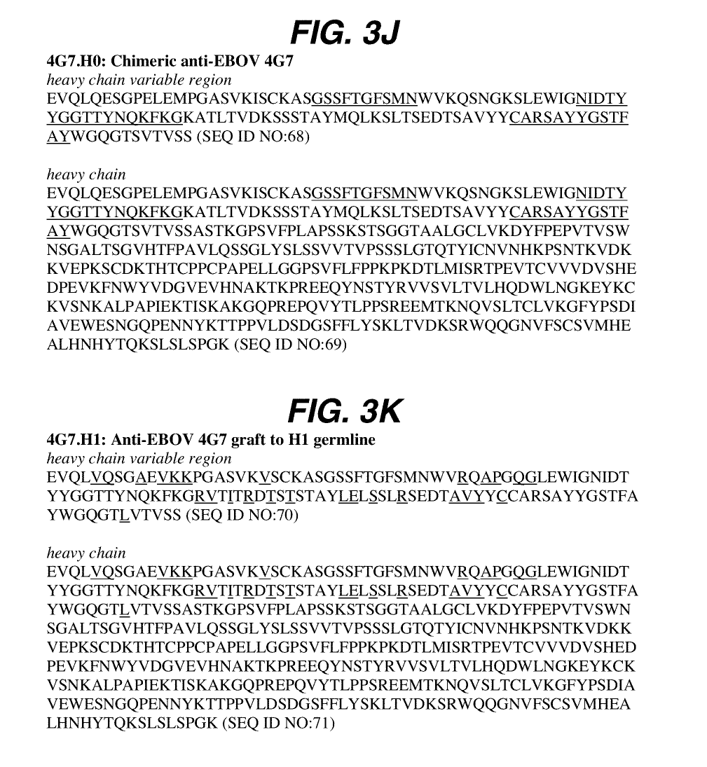

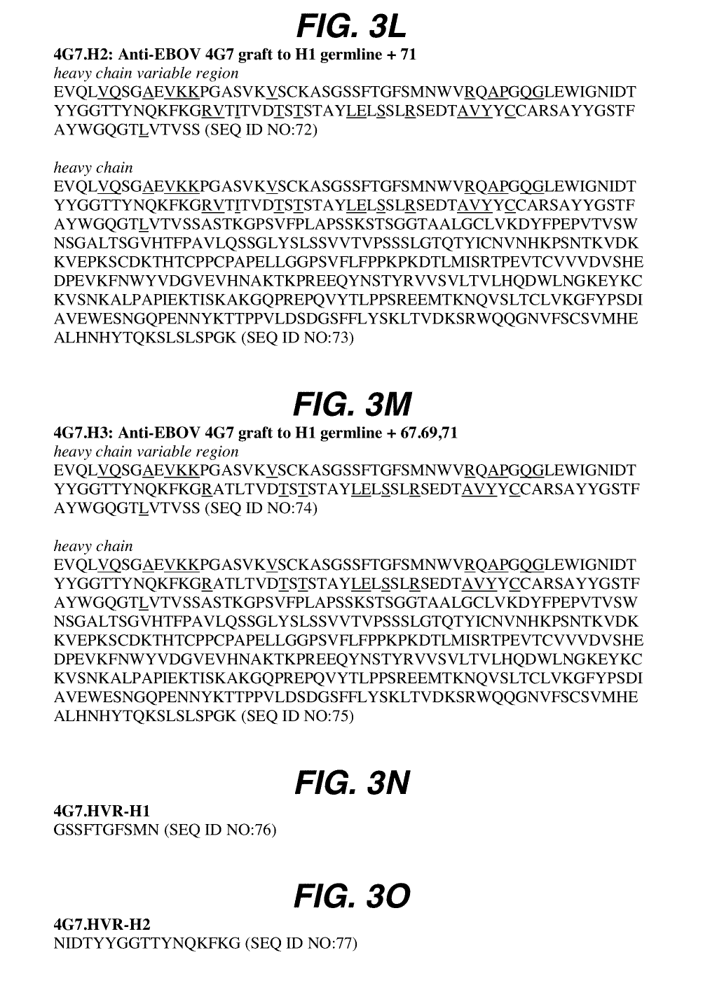

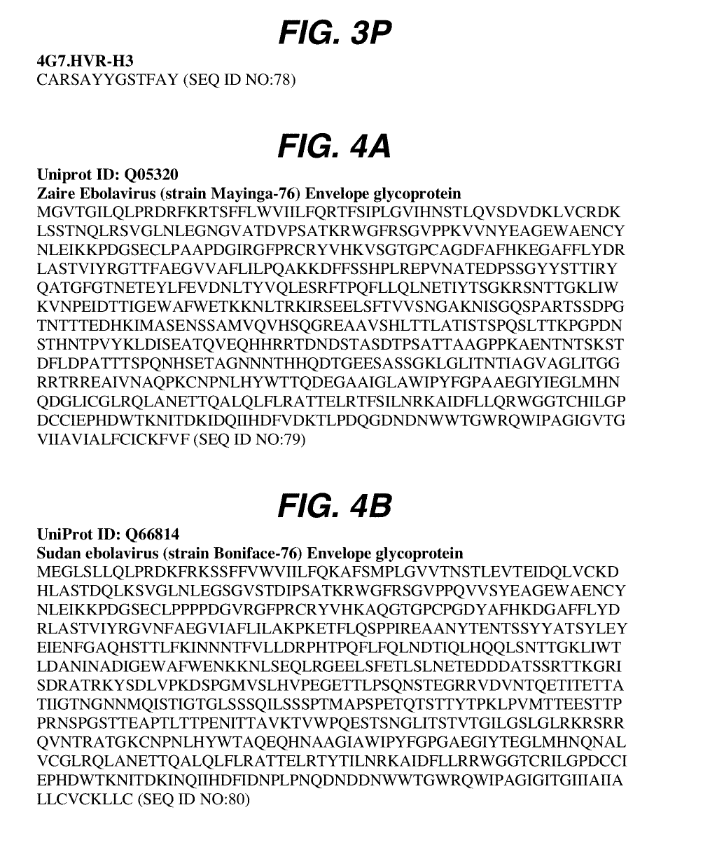

In certain embodiments, HVR residues comprise those identified in FIG. 1E, FIG. 1F, and FIG. 1G (SEQ ID NOS:9, 10, 11); FIG. 1M, FIG. 1N, and FIG. 1O (SEQ ID NOS:22, 23, 24); FIG. 2G, FIG. 2H, and FIG. 2I (SEQ ID NOS:37, 38, 39); FIG. 2O, FIG. P, and FIG. 2Q (SEQ ID NOS:50, 51, 52); FIG. 3G, FIG. 3H, and FIG. 3I (SEQ ID NOS:65, 66, 67); and FIG. 3N, FIG. 3O, and FIG. 3P (SEQ ID NOS:76, 77, 78).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-Ebola virus glycoprotein antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “Ebola virus glycoprotein,” or “Ebola virus envelop glycoprotein,” as used herein, refers to any native Ebola virus glycoprotein from any Ebola virus strain or isolate. The term encompasses “full-length,” unprocessed Ebola virus glycoprotein as well as any form of Ebola virus glycoprotein that results from processing in the Ebola virus or in a cell infected with Ebola virus. The term also encompasses naturally occurring variants of Ebola virus glycoprotein, e.g., splice variants or allelic variants. The amino acid sequences of exemplary Ebola virus glycoproteins are shown in FIG. 4A-FIG. 4L (SEQ ID NOS:79-90). Ebola virus glycoproteins are further described in, e.g., Lee et al., (2009) Future Virol. 4(6):621-635, Lee et al., (2008) Nature 454:177-182, WO 2005/063798, WO 2011/071574).

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### II. Compositions and Methods

In one aspect, the invention is based, in part, on anti-Ebola virus GP antibodies. In certain embodiments, humanized antibodies that bind to Ebola virus glycoprotein are provided. Antibodies of the invention are useful, e.g., for the diagnosis or treatment of Ebola virus disease.

A. Exemplary Anti-Ebola Virus Glycoprotein Antibodies

In one aspect, the invention provides isolated antibodies that bind to Ebola virus glycoprotein. In certain embodiments, a humanized anti-Ebola virus glycoprotein antibody is provided, e.g., a humanized antibody that binds to an epitope comprising the amino acid sequence of SEQ ID NO:91 or SEQ ID NO:92.

In one aspect, the invention provides a humanized anti-Ebola virus glycoprotein antibody comprising at least one, two, three, four, five, or six HVRs selected from (a) HVR-H1 comprising the amino acid sequence of SEQ ID NOS:22, 50, or 76; (b) HVR-H2 comprising the amino acid sequence of SEQ ID NOS:23, 51, or 77; (c) HVR-H3 comprising the amino acid sequence of SEQ ID NOS:24, 52, or 78; (d) HVR-L1 comprising the amino acid sequence of SEQ ID NOS:9, 37, or 65; (e) HVR-L2 comprising the amino acid sequence of SEQ ID NOS:10, 38, or 66; and (f) HVR-L3 comprising the amino acid sequence of SEQ ID NOS:11, 39, or 67.

In another aspect, an antibody of the invention comprises (a) a VH domain comprising at least one, at least two, or all three VH HVR sequences selected from (i) HVR-H1 comprising the amino acid sequence of SEQ ID NO:22, (ii) HVR-H2 comprising the amino acid sequence of SEQ ID NO:23, and (iii) HVR-H3 comprising an amino acid sequence selected from SEQ ID NO:24; and (b) a VL domain comprising at least one, at least two, or all three VL HVR sequences selected from (i) HVR-L1 comprising the amino acid sequence of SEQ ID NO:9, (ii) HVR-L2 comprising the amino acid sequence of SEQ ID NO:10, and (c) HVR-L3 comprising the amino acid sequence of SEQ ID NO:11. In another aspect, the invention provides an antibody comprising (a) HVR-H1 comprising the amino acid sequence of SEQ ID NO:22; (b) HVR-H2 comprising the amino acid sequence of SEQ ID NO:23; (c) HVR-H3 comprising the amino acid sequence of SEQ ID NO:24; (d) HVR-L1 comprising the amino acid sequence of SEQ ID NO:9; (e) HVR-L2 comprising the amino acid sequence of SEQ ID NO:10; and (f) HVR-L3 comprising an amino acid sequence selected from SEQ ID NO:11.

In another aspect, an antibody of the invention comprises (a) a VH domain comprising at least one, at least two, or all three VH HVR sequences selected from (i) HVR-H1 comprising the amino acid sequence of SEQ ID NO:50, (ii) HVR-H2 comprising the amino acid sequence of SEQ ID NO:51, and (iii) HVR-H3 comprising an amino acid sequence selected from SEQ ID NO:52; and (b) a VL domain comprising at least one, at least two, or all three VL HVR sequences selected from (i) HVR-L1 comprising the amino acid sequence of SEQ ID NO:37, (ii) HVR-L2 comprising the amino acid sequence of SEQ ID NO:38, and (c) HVR-L3 comprising the amino acid sequence of SEQ ID NO:39. In another aspect, the invention provides an antibody comprising (a) HVR-H1 comprising the amino acid sequence of SEQ ID NO:50; (b) HVR-H2 comprising the amino acid sequence of SEQ ID NO:51; (c) HVR-H3 comprising the amino acid sequence of SEQ ID NO:52; (d) HVR-L1 comprising the amino acid sequence of SEQ ID NO:37; (e) HVR-L2 comprising the amino acid sequence of SEQ ID NO:38; and (f) HVR-L3 comprising an amino acid sequence selected from SEQ ID NO:39.

In another aspect, an antibody of the invention comprises (a) a VH domain comprising at least one, at least two, or all three VH HVR sequences selected from (i) HVR-H1 comprising the amino acid sequence of SEQ ID NO:76, (ii) HVR-H2 comprising the amino acid sequence of SEQ ID NO:77, and (iii) HVR-H3 comprising an amino acid sequence selected from SEQ ID NO:78; and (b) a VL domain comprising at least one, at least two, or all three VL HVR sequences selected from (i) HVR-L1 comprising the amino acid sequence of SEQ ID NO:65, (ii) HVR-L2 comprising the amino acid sequence of SEQ ID NO:66, and (c) HVR-L3 comprising the amino acid sequence of SEQ ID NO:67. In another aspect, the invention provides an antibody comprising (a) HVR-H1 comprising the amino acid sequence of SEQ ID NO:76; (b) HVR-H2 comprising the amino acid sequence of SEQ ID NO:77; (c) HVR-H3 comprising the amino acid sequence of SEQ ID NO:78; (d) HVR-L1 comprising the amino acid sequence of SEQ ID NO:65; (e) HVR-L2 comprising the amino acid sequence of SEQ ID NO:66; and (f) HVR-L3 comprising an amino acid sequence selected from SEQ ID NO:67.

In certain embodiments, any one or more amino acids in the anti-Ebola virus glycoprotein antibodies as provided above are substituted at the following HVR positions (Kabat numbering):

In 13C6.HVR-H1 (SEQ ID NO: 22): position 35b

In 13C6.HVR-H2 (SEQ ID NO:23): position 52, 54, 58, 60, or 61

In 13C6.HVR-H3 (SEQ ID NO:24): position 100d or 101

In 13C6.HVR-L1 (SEQ ID NO:9): position 24, 28, 29, 30, 31, 32, or 33

In 13C6.HVE-L2 (SEQ ID NO:10): position 50, 53, 54, 55, or 56

In 13C6.HVR-L3 (SEQ ID NO:11): position 92 or 96

In 2G4.HVR-H1 (SEQ ID NO:50): position 31, 33, or 35

In 2G4.HVR-H2 (SEQ ID NO:51): position 50, 52, 52a, 52b, 52c, 53, 54, 55, 56, 58, or

In 2G4.HVR-H3 (SEQ ID NO:52): position 93 or 100b

In 2G4.HVR-L1 (SEQ ID NO:37): position 27, 28, 30, or 32

In 2G4.HVR-L2 (SEQ ID NO:38): position 50, 52, 53, 55, or 56

In 2G4.HVR-L3 (SEQ ID NO:39): position 90, 91, 92, 93, 94, or 96

In 4G7.HVR-H1 (SEQ ID NO:76): position 27, 28, 31, 32, 33, 34, or 35

In 4G7.HVR-H2 (SEQ ID NO:77): position 50, 52, 52a, 53, 54, 56, 58, 60, or 64

In 4G7.HVR-H3 (SEQ ID NO:78): position 93, 94, or 101

In 4G7.HVR-L1 (SEQ ID NO:65): position 27, 28, or 30

In 4G7.HVR-L2 (SEQ ID NO:66): position 50, 52, 53, 55, or 56

In 4G7.HVR-L3 (SEQ ID NO:67): position 90, 91, 92, 93, or 94

In certain embodiments, the substitutions are conservative substitutions, as provided herein.

In certain embodiments, an anti-Ebola virus glycoprotein antibody of the invention is as described in Tables 1-3.

In any of the above embodiments, the anti-Ebola virus glycoprotein antibody is humanized. In one embodiment, the anti-Ebola virus glycoprotein antibody comprises HVRs as in any of the above embodiments, and further comprises an acceptor human framework, e.g., a human immunoglobulin framework or a human consensus framework.

In another aspect, an anti-Ebola virus glycoprotein antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NOS:14, 42, or 70. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-Ebola virus glycoprotein antibody comprising that sequence retains the ability to bind to Ebola virus glycoprotein. In certain embodiments, a total of 1 to 10 amino acids have been substituted, inserted and/or deleted in SEQ ID NOS:14, 42, or 70. In certain embodiments, substitutions, insertions, or deletions occur in regions outside the HVRs (i.e., in the FRs, e.g., in the heavy chain Vernier positions 2, 24, 37, 49, 66, 67, 68, 69, 71, 73, and/or 75 (Kabat numbering)). In some embodiments, the anti-Ebola virus glycoprotein antibody comprises the VH (variable heavy chain region) sequence in SEQ ID NOS:14, 16, 18, 20, 42, 44, 46, 48, 70, 72, or 74, including post-translational modifications of that sequence. In a particular embodiment, the VH comprises one, two or three HVRs selected from: (a) HVR-H1 comprising the amino acid sequence of SEQ ID NOS:22, 50, or 76, (b) HVR-H2 comprising the amino acid sequence of SEQ ID NOS:23, 51, or 77, and (c) HVR-H3 comprising the amino acid sequence of SEQ ID NOS:24, 52, or 78.

In another aspect, an anti-Ebola virus glycoprotein antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NOS:3, 27, or 55. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but a humanized anti-Ebola virus glycoprotein antibody comprising that sequence retains the ability to bind to Ebola virus glycoprotein. In certain embodiments, a total of 1 to 10 amino acids have been substituted, inserted and/or deleted in SEQ ID NOS:3, 27, or 55. In certain embodiments, the substitutions, insertions, or deletions occur in regions outside the HVRs (i.e., in the FRs, e.g., in the light chain Vernier positions 4, 43, 48, 71, and/or 87 (Kabat numbering)). In some embodiments, the anti-Ebola virus glycoprotein antibody comprises the

VL sequence in SEQ ID NOS:3, 5, 7, 27, 29, 31, 33, 35, 55, 57, 59, 61, or 63, including post-translational modifications of that sequence. In a particular embodiment, the VL comprises one, two or three HVRs selected from (a) HVR-L1 comprising the amino acid sequence of SEQ ID NOS:9, 37, or 65; (b) HVR-L2 comprising the amino acid sequence of SEQ ID NOS:10, 38, or 66; and (c) HVR-L3 comprising the amino acid sequence of SEQ ID NOS:11, 39, or 67.

In another aspect, an anti-Ebola virus glycoprotein antibody is provided, wherein the antibody comprises a VH as in any of the embodiments provided above, and a VL as in any of the embodiments provided above. Exemplary antibodies comprising particular VH (variable heavy chain) and VL (Variable light chain) sequences or particular heavy chain and light chain sequences are detailed in Tables 1-3 below.

TABLE 1
Humanized 13C6 antibody variants

| Variant | Designation | VL Sequence | Light Chain Sequence | VH Sequence | Heavy Chain Sequence |
|---|---|---|---|---|---|
| 1 | h13C6a | SEQ ID NO: 3 | SEQ ID NO: 4 | SEQ ID NO: 14 | SEQ ID NO: 15 |
| 2 | h13C6b | SEQ ID NO: 3 | SEQ ID NO: 4 | SEQ ID NO: 20 | SEQ ID NO: 21 |
| 3 | h13C6c | SEQ ID NO: 3 | SEQ ID NO: 4 | SEQ ID NO: 18 | SEQ ID NO: 19 |
| 4 | h13C6d | SEQ ID NO: 3 | SEQ ID NO: 4 | SEQ ID NO: 16 | SEQ ID NO: 17 |
| 5 | h13C6e | SEQ ID NO: 5 | SEQ ID NO: 6 | SEQ ID NO: 14 | SEQ ID NO: 15 |
| 6 | h13C6f | SEQ ID NO: 5 | SEQ ID NO: 6 | SEQ ID NO: 20 | SEQ ID NO: 21 |
| 7 | h13C6g | SEQ ID NO: 5 | SEQ ID NO: 6 | SEQ ID NO: 18 | SEQ ID NO: 19 |
| 8 | h13C6h | SEQ ID NO: 5 | SEQ ID NO: 6 | SEQ ID NO: 16 | SEQ ID NO: 17 |
| 9 | h13C6i | SEQ ID NO: 7 | SEQ ID NO: 8 | SEQ ID NO: 14 | SEQ ID NO: 15 |
| 10 | h13C6j | SEQ ID NO: 7 | SEQ ID NO: 8 | SEQ ID NO: 20 | SEQ ID NO: 21 |
| 11 | h13C6k | SEQ ID NO: 7 | SEQ ID NO: 8 | SEQ ID NO: 18 | SEQ ID NO: 19 |
| 12 | h13C6l | SEQ ID NO: 7 | SEQ ID NO: 8 | SEQ ID NO: 16 | SEQ ID NO: 17 |

TABLE 2
Humanized 2G4 antibody variants

| | | | Light Chain | | Heavy Chain |
|---|---|---|---|---|---|
| Variant | Designation | VL Sequence | Sequence | VH Sequence | Sequence |
| 13 | h2G4a | SEQ ID | SEQ ID | SEQ ID NO: 42 | SEQ ID NO: 43 |
| | | NO: 27 | NO: 28 | | |
| 14 | h2G4b | SEQ ID NO: 27 | SEQ ID | SEQ ID NO: 44 | SEQ ID NO: 45 |
| | | | NO: 28 | | |
| 15 | h2G4c | SEQ ID NO: 27 | SEQ ID | SEQ ID NO: 46 | SEQ ID NO: 47 |
| | | | NO: 28 | | |
| 16 | h2G4d | SEQ ID NO: 27 | SEQ ID | SEQ ID NO: 48 | SEQ ID NO: 49 |
| | | | NO: 28 | | |
| 17 | h2G4e | SEQ ID NO: 29 | SEQ ID | SEQ ID NO: 42 | SEQ ID NO: 43 |
| | | | NO: 30 | | |
| 18 | h2G4f | SEQ ID NO: 29 | SEQ ID | SEQ ID NO: 44 | SEQ ID NO: 45 |
| | | | NO: 30 | | |
| 19 | h2G4g | SEQ ID NO: 29 | SEQ ID | SEQ ID NO: 46 | SEQ ID NO: 47 |
| | | | NO: 30 | | |
| 20 | h2G4h | SEQ ID NO: 29 | SEQ ID | SEQ ID NO: 48 | SEQ ID NO: 49 |
| | | | NO: 30 | | |
| 21 | h2G4i | SEQ ID NO: 31 | SEQ ID | SEQ ID NO: 42 | SEQ ID NO: 43 |
| | | | NO: 32 | | |
| 22 | h2G4j | SEQ ID NO: 31 | SEQ ID | SEQ ID NO: 44 | SEQ ID NO: 45 |
| | | | NO: 32 | | |
| 23 | h2G4k | SEQ ID NO: 31 | SEQ ID | SEQ ID NO: 46 | SEQ ID NO: 47 |
| | | | NO: 32 | | |
| 24 | h2G4l | SEQ ID NO: 31 | SEQ ID | SEQ ID NO: 48 | SEQ ID NO: 49 |
| | | | NO: 32 | | |
| 25 | h2G4m | SEQ ID NO: 33 | SEQ ID | SEQ ID NO: 48 | SEQ ID NO: 49 |
| | | | NO: 34 | | |
| 26 | h2G4n | SEQ ID NO: 35 | SEQ ID | SEQ ID NO: 42 | SEQ ID NO: 43 |
| | | | NO: 36 | | |
| 27 | h2G4o | SEQ ID NO: 35 | SEQ ID | SEQ ID NO: 44 | SEQ ID NO: 45 |
| | | | NO: 36 | | |
| 28 | h2G4p | SEQ ID NO: 35 | SEQ ID | SEQ ID NO: 48 | SEQ ID NO: 47 |
| | | | NO: 36 | | |
| 29 | h2G4q | SEQ ID NO: 33 | SEQ ID | SEQ ID NO: 42 | SEQ ID NO: 43 |
| | | | NO: 34 | | |
| 30 | h2G4r | SEQ ID NO: 33 | SEQ ID | SEQ ID NO: 44 | SEQ ID NO: 45 |
| | | | NO: 34 | | |
| 31 | h2G4s | SEQ ID NO: 33 | SEQ ID | SEQ ID NO: 46 | SEQ ID NO: 47 |
| | | | NO: 34 | | |
| 32 | h2G4t | SEQ ID NO: 35 | SEQ ID | SEQ ID NO: 46 | SEQ ID NO: 47 |
| | | | NO: 36 | | |

TABLE 3
Humanized 4G7 antibody variants

| | | | Light Chain | | Heavy Chain |
|---|---|---|---|---|---|
| Variant | Designation | VL Sequence | Sequence | VH Sequence | Sequence |
| 33 | h4G7b | SEQ ID | SEQ ID | SEQ ID NO: 70 | SEQ ID NO: 71 |
| | | NO: 55 | NO: 56 | | |
| 34 | h4G7c | SEQ ID NO: 55 | SEQ ID | SEQ ID NO: 74 | SEQ ID NO: 75 |
| | | | NO: 56 | | |
| 35 | h4G7d | SEQ ID NO: 55 | SEQ ID | SEQ ID NO: 72 | SEQ ID NO: 73 |
| | | | NO: 56 | | |
| 36 | h4G7e | SEQ ID NO: 57 | SEQ ID | SEQ ID NO: 70 | SEQ ID NO: 71 |
| | | | NO: 58 | | |
| 37 | h4G7f | SEQ ID NO: 57 | SEQ ID | SEQ ID NO: 74 | SEQ ID NO: 75 |
| | | | NO: 58 | | |
| 38 | h4G7g | SEQ ID | SEQ ID | SEQ ID NO: 72 | SEQ ID NO: 73 |
| | | NO: 57 | NO: 58 | | |
| 39 | h4G7h | SEQ ID NO: 61 | SEQ ID | SEQ ID NO: 70 | SEQ ID NO: 71 |
| | | | NO: 62 | | |
| 40 | h4G7i | SEQ ID NO: 61 | SEQ ID | SEQ ID NO: 74 | SEQ ID NO: 75 |
| | | | NO: 62 | | |
| 41 | h4G7j | SEQ ID NO: 61 | SEQ ID | SEQ ID NO: 72 | SEQ ID NO: 73 |
| | | | NO: 62 | | |
| 42 | h4G7k | SEQ ID NO: 63 | SEQ ID | SEQ ID NO: 70 | SEQ ID NO: 71 |
| | | | NO: 64 | | |
| 43 | h4G7l | SEQ ID NO: 63 | SEQ ID | SEQ ID NO: 74 | SEQ ID NO: 75 |
| | | | NO: 64 | | |
| 44 | h4G7m | SEQ ID NO: 63 | SEQ ID | SEQ ID NO: 72 | SEQ ID NO: 73 |
| | | | NO: 64 | | |
| 45 | h4G7n | SEQ ID NO: 59 | SEQ ID | SEQ ID NO: 70 | SEQ ID NO: 71 |
| | | | NO: 60 | | |
| 46 | h4G7o | SEQ ID NO: 59 | SEQ ID | SEQ ID NO: 74 | SEQ ID NO: 75 |
| | | | NO: 60 | | |
| 47 | h4G7p | SEQ ID NO: 59 | SEQ ID | SEQ ID NO: 72 | SEQ ID NO: 73 |
| | | | NO: 60 | | |

In a particular embodiment, the antibody comprises the VH and VL sequences in SEQ ID NO:14 and SEQ ID NO:7, respectively, including post-translational modifications of those sequences. In another particular embodiment, the antibody comprises the VH and VL sequences in SEQ ID NO:42 and SEQ ID NO:35, respectively, including post-translational modifications of those sequences. In yet another particular embodiment, the antibody comprises the VH and VL sequences in SEQ ID NO:70 and SEQ ID NO:55, respectively, including post-translational modifications of those sequences.

In a further aspect, the invention provides an antibody that binds to the same epitope as an anti-Ebola virus glycoprotein antibody provided herein. For example, in certain embodiments, an antibody is provided that binds to the same epitope as an anti-Ebola virus glycoprotein antibody comprising a VH sequence of SEQ ID NO:12 and a VL sequence of SEQ ID NO:1. In other embodiments, an antibody is provided that binds the same epitope as an anti-Ebola virus glycoprotein antibody comprising a VH sequence of SEQ ID NO:40 and a VL sequence of SEQ ID NO:25. In yet other embodiments, an antibody is provided that binds the same epitope as an anti-Ebola virus glycoprotein antibody comprising a VH sequence of SEQ ID NO:68 and a VL sequence of SEQ ID NO:53. In certain embodiments, an antibody is provided that binds to an epitope within a fragment of the glycoprotein gene of Ebola Zaire (e.g., GenBank accession number L11365) consisting of amino acids 389-493 (SEQ ID NO:91) (see also, e.g., U.S. Pat. No. 7,335,356, describing the epitope recognized by anti-Ebola virus glycoprotein antibody 13C6. In other embodiments, an antibody is provided that binds to an epitope within a fragement of Ebola Zaire envelope glycoprotein (e.g., UniProt ID: P87666) consisting of amino acids 502-516 (SEQ ID NO:92) (see also, e.g., U.S. Pat. No. 8,513,391 describing epitope mapping studies of various monoclonal antibodies).

In a further aspect of the invention, an anti-Ebola virus glycoprotein antibody according to any of the above embodiments is a monoclonal antibody, including a chimeric, humanized or human antibody. In one embodiment, an anti-Ebola virus glycoprotein antibody is an antibody fragment, e.g., a Fv, Fab, Fab′, scFv, diabody, or F(ab′)₂ fragment. In another embodiment, the antibody is a full length antibody, e.g., an intact antibody or other antibody class or isotype (e.g., IgA1, IgA2, IgD, IgE, IgG1, IgG2, IgG3, IgG4, or IgM) as defined herein.

In a further aspect, an anti-Ebola virus glycoprotein antibody according to any of the above embodiments may incorporate any of the features, singly or in combination, as described in Sections 1-7 below:

### 1. Antibody Affinity

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵1]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Strategies for identifying residues in the FR amenable to substitution are as follows: (a) analysis of donor and acceptor sequences: putative point backmutations from the human framework residue back to the original source residues can be identified from the analysis of the donor and acceptor sequences for canonical residues, interchain packing residues, unusual residues and glycoslyation sites. In particular embodiments, it has shown to be important to retain the source's canonical and interchain packing residues (e.g., see antibodies 60.3 and BR96); (b) analysis of Vernier Zone and CDR-H3: residues in the Vernier zone which form a “platform” on which the CDRs rest and therefore potentially affect their conformation can be substituted. Due to their extreme variability, there are no canonical residues defined for CDR-H3, therefore attention should be paid to this loop to analyze the structural model for residues which may potentially affect its conformation; (c) analysis of proximity to binding site: if a structural model is available, an analysis of the residues within 5 angstroms of any CDR residue should be examined as these residues are likely to bind to the antigen especially if these residues are classified as important by the analysis of the source sequence; (d) analysis of glycosylation sites: consider whether removal of potential glycoslyation sites, especially if present on the surface of an available structural model of the antibody, since removal of glycosylation often enhances the avidity of the humanized antibody.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al, Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J Mol. Biol. 338(2): 299-310 (2004); Lee et al., J Mol. Biol. 340(5): 1073-1093 (2004); Fellouse, Proc. Natl. Acad. Sci. USA 101(34): 12467-12472 (2004); and Lee et al., J Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for Ebola virus glycoprotein and the other is for any other antigen. In certain embodiments, bispecific antibodies may bind to two different epitopes of Ebola virus glycoprotein. Bispecific antibodies may also be used to localize cytotoxic agents to Ebola virus or to cells infected with Ebola virus which express Ebola virus glycoprotein. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to Ebola virus glycoprotein as well as another, different antigen (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 4 under the heading of “preferred substitutions.” More substantial changes are provided in Table 4 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 4

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region or has reduced fucosylation. Robust and stable production of fully non-fucosylated therapeutic antibodies with fixed quality is required for the development of therapeutic antibodies because the high level of ADCC efficacy of non-fucosylated therapeutic antibody molecules is reduced in vivo by fucosylated counterparts through competition for binding to the antigen on target cells (see, e.g., Yamane-Ohnuki et al., (2009) Mabs. 1(3):230-236, Mori et al., (2007) Cytotechnology. 55(2-3):109-114). The present invention utilizes mammalian host cell lines that can stably produce fully non-fucosylated antibodies and these antibodies possess beneficial characteristics detailed in Examples 1-4 below. In various aspects, the invention also features the production of antibodies with controlled fucosylation.

In particular embodiments, for example, the amount of fucose in such antibody may be from 0% to 1%, 0% to 2%, 1% to 80%, from 1% to 65%, from 5% to 65%, or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated (or afucosylated) antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

B. Recombinant Methods and Compositions

Antibodies may be produced using recombinant methods and compositions, e.g., as described in U.S. Pat. No. 4,816,567. In one embodiment, isolated nucleic acid encoding an anti-Ebola virus glycoprotein antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, the host cell is a plant cell, e.g. a tobacco plant cell (e.g., Tobacco BY-2 cells, see, e.g., Nagata et al., (1992) Inter. Rev. Cyto 132:1-30, Kirchhoff et al., (2012) Plant Biotechnol. J. 10.8:936-944). In other embodiments, the host cell is a cell capable of producing defucosylated (or afucosylated) antibodies, such as Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107). In one embodiment, a method of making an anti-Ebola virus glycoprotein antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-Ebola virus glycoprotein antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants), Fischer et al., (2003) Vaccine 21:820-825 (describing production of antibodies in plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. Other useful mammalian host cell lines also include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107). For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

C. Assays

Anti-Ebola virus glycoprotein antibodies provided herein may be identified, screened for, or characterized for their physical/chemical properties and/or biological activities by various assays known in the art.

### 1. Binding Assays and Other Assays

In one aspect, an antibody of the invention is tested for its antigen binding activity, e.g., by known methods such as ELISA, Western blot, etc.

In another aspect, competition assays may be used to identify an antibody that competes with one or more of the humanized anti-Ebola antibodies having a VH and VL sequence, or a heavy chain and light chain sequence, as listed in Tables 1-3 for binding to Ebola virus glycoprotein. In certain embodiments, such a competing antibody binds to the same epitope (e.g., a linear or a conformational epitope) that is bound by one or more of the humanized anti-Ebola antibodies having a VH and VL sequence, or a heavy chain and light chain sequence, as listed in Tables 1-3. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized Ebola virus glycoprotein is incubated in a solution comprising a first labeled antibody that binds to Ebola virus glycoprotein (e.g., one or more of the humanized anti-Ebola antibodies having a VH and VL sequence, or heavy chain and light chain sequence, as listed in Tables 1-3) and a second unlabeled antibody that is being tested for its ability to compete with the first antibody for binding to Ebola virus glycoprotein. The second antibody may be present in a hybridoma supernatant. As a control, immobilized Ebola virus glycoprotein is incubated in a solution comprising the first labeled antibody but not the second unlabeled antibody. After incubation under conditions permissive for binding of the first antibody to Ebola virus glycoprotein, excess unbound antibody is removed, and the amount of label associated with immobilized Ebola virus glycoprotein is measured. If the amount of label associated with immobilized Ebola virus glycoprotein is substantially reduced in the test sample relative to the control sample, then that indicates that the second antibody is competing with the first antibody for binding to Ebola virus glycoprotein. See Harlow and Lane (1988) Antibodies: A Laboratory Manual ch. 14 (Cold Spring Harbor Laboratory, Cold Spring Harbor, N.Y.).

### 2. Activity Assays

In one aspect, assays are provided for identifying anti-Ebola virus glycoprotein antibodies having biological activity. Biological activity may include, e.g., inhibition of plaque formation by Ebola virus, protection against infection, and/or extended survival post infection

In certain embodiments, an antibody of the invention is tested for such biological activity by, for example, an in vitro plaque-reduction neutralization assay. Plaque assays can be performed e.g., using confluent Vero-E6 cells. To evaluate the presence of Ebola virus-neutralizing antibodies, dilutions of the antibodies can be mixed with 100 pfu of mouse-adapted Ebola virus at 37° C. for 1 h, and used to infect Vero E6 cells. Cells are then covered with an agarose overlay (Moe, J. et al. (1981) J. Clin. Microbiol, 13:791-793) and a second overlay containing 5% neutral red solution in PBS or agarose is added 6 days later. Plaques are then counted the following day. Endpoint titers are determined to be the last dilution of antibody that reduced the number of plaques by 80% of the control wells. For further details of in vitro plaque-reduction neutralization assays, see, e.g., WO 2010/016183.

In other embodiments, antibodies of the invention can also be tested for in vivo biological activity (e.g., protection against infection, and/or extended survival post infection). For example, to determine the prophylactic and/or therapeutic benefit of the anti-Ebola virus glycoprotein antibodies of the invention, purified antibodies or combinations of antibodies can be injected intraperitoneally into BALB/c or C57BL/6 mice 2.4 h prior to challenge with mouse-adapted Ebola Zaire virus, and/or either 1, 2, or 3 days after challenge with mouse-adapted Ebola Zaire virus. Ebola infection in mice can be performed by intraperitoneal inoculation of 10 pfu of mouse-adapted Ebola Zaire 1976 virus. The animals are then monitored for morbidity and mortality for 28 days post-infection, with and without treatment of the anti-Ebola viral glycoprotein antibodies (for further details on mouse studies, see, e.g., WO 2010/016183). For additional in vivo biological assays in primates and guinea pigs, see, e.g., Qiu et al., (2014) Nature 514:47-53, U.S. Pat. No. 8,513,391.

D. Immunoconjugates

The invention also provides immunoconjugates comprising an anti-Ebola virus glycoprotein antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinyl sulfone)benzoate) which are commercially available (e.g., from Pierce Biotechnology, Inc., Rockford, Ill., U.S.A).

E. Methods and Compositions for Diagnostics and Detection

In certain embodiments, any of the anti-Ebola virus glycoprotein antibodies provided herein is useful for detecting the presence of Ebola virus glycoprotein in a biological sample.

The term “detecting” as used herein encompasses quantitative or qualitative detection. In certain embodiments, a biological sample comprises e.g., whole blood in EDTA, serum, nasal fluid, or oral swabs.

In one embodiment, an anti-Ebola virus glycoprotein antibody for use in a method of diagnosis or detection is provided. In a further aspect, a method of detecting the presence of

Ebola virus glycoprotein in a biological sample is provided. In certain embodiments, the method comprises contacting the biological sample with an anti-Ebola virus glycoprotein antibody as described herein under conditions permissive for binding of the anti-Ebola virus glycoprotein antibody to Ebola virus glycoprotein, and detecting whether a complex is formed between the anti-Ebola virus glycoprotein antibody and Ebola virus glycoprotein. Such method may be an in vitro or in vivo method. In one embodiment, an anti-Ebola virus glycoprotein antibody is used to select subjects eligible for therapy with an anti-Ebola virus glycoprotein antibody, e.g. where Ebola virus glycoprotein is a biomarker for selection of patients.

Other methods of diagnosis or detection include: (a) basic blood tests: the early phase of infection is characterized by thrombocytopenia, leukopenia, and a pronounced lymphopenia. Neutrophilia develops after several days, as do elevations in aspartate aminotransferase and alanine aminotransferase. Bilirubin may be normal or slightly elevated. With the onset of anuria, blood urea nitrogen and serum creatinine increase. Terminally ill patients may develop a metabolic acidosis that may contribute to the observation that these patients often have tachypnea, which may be an attempt at compensatory hyperventilation; (b) studies for isolating virus: definitive diagnosis rests on isolation of the virus by means of tissue culture or reverse-transcription polymerase chain reaction (RT-PCR) assay. Isolation of Ebola virus in tissue culture is a high-risk procedure that can be performed safely only in a few high-containment laboratories throughout the world; (c) serologic testing for antibody and antigen: the indirect fluorescence antibody test (IFAT) is associated with false-positive results. Concerns over the sensitivity and utility of this test have resulted in the development of confirmatory serologic tests. In infected patients who survive long enough to develop an immune response, the immunoglobulin M (IgM) and immunoglobulin G (IgG) enzyme-linked immunosorbent assay (ELISA) tests may be useful in the diagnosis of Ebola virus infection. Both ELISA tests have been demonstrated to be sensitive and specific. IgM-capture ELISA uses Zaire ebolavirus antigens grown in Vero E6 cells to detect IgM antibodies to this strain. Results become positive in experimental primates within 6 days of infection but do not remain positive for extended periods. These qualities indicate that the IgM test may be used to document acute Ebola infection. IgG-capture ELISA uses detergent-extracted viral antigens to detect IgG anti-Ebola antibodies. It is more specific than the IFAT, and it remains positive for long periods. An antigen detection ELISA test is available that identifies Ebola virus antigens. Other methods used to confirm the diagnosis of Ebola virus infection include an immunohistochemical test performed on formalin-fixed postmortem skin taken from patients who have died of Ebola hemorrhagic fever.

Exemplary disorders that may be diagnosed using an antibody of the invention include Ebola virus disease and Ebola virus infection.

In certain embodiments, labeled anti-Ebola virus glycoprotein antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

F. Pharmaceutical Formulations

Pharmaceutical formulations of an anti-Ebola virus glycoprotein antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide, for example, another anti-Ebola virus antibody (e.g., 5D2, 5E6, 6D3, 6D8, 7C9, 7G4, 1H3, 1008, 12B5, or 13F6), an anti-viral agent (e.g., adamantine antivirals, antiviral interferons, chemokine receptor antagonist, integrase strand transfer inhibitor, neuraminidase inhibitors, NNRTIs, protease inhibitors, purine nucleosides, or nucleoside reverse transcriptase inhibitors), etc. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

G. Therapeutic Methods and Compositions

Any of the anti-Ebola virus glycoprotein antibodies provided herein may be used in therapeutic methods.

In one aspect, an anti-Ebola virus glycoprotein antibody for use as a medicament is provided. In further aspects, an anti-Ebola virus glycoprotein antibody for use in treating Ebola virus disease or infection is provided. In certain embodiments, an anti-Ebola virus glycoprotein antibody for use in a method of treatment is provided. In certain embodiments, the invention provides an anti-Ebola virus glycoprotein antibody for use in a method of treating an individual having Ebola virus disease or infection comprising administering to the individual an effective amount of one or more anti-Ebola virus glycoprotein antibodies. In one such embodiment, the method comprises administering to the individual an effective amount of an anti-Ebola virus glycoprotein antibody as described in Table 1 (h13C6a, h13C6b, h13C6c, h13C6d, h13C6e, h13C6f, h13C6g, h13C6h, h13C6i, h13C6j, h13C6k, or h13C61). In another such embodiment, the method comprises administering to the individual an effective amount of an anti-Ebola virus glycoprotein antibody as described in Table 2 (h2G4a, h2G4b, h2G4c, h2G4d, h2G4e, h2G4f, h2G4g, h2G4b, h2G4i, h2G4j, h2G4k, h2G41, h2G4m, h2G4n, h2G4o, h2G4p, h2G4q, h2G4r, h2G4s, or h2G4t). In another such embodiment, the method comprises administering to the individual an effective amount of an anti-Ebola virus glycoprotein antibody as described in Table 3 (h4G7b, h4G7c, h4G7d, h4G7e, h4G7f, h4G7g, h4G7h, h4G7i, h4G7j, h4G7k, h4G71, h4G7m, h4G7n, h4G7o, or h4G7p). In yet other embodiments, the method comprises administering to the individual an effective amount of an anti-Ebola virus glycoprotein antibody as described in Table 1, an effective amount of an anti-Ebola virus glycoprotein antibody as described in Table 2, and an effective amount of an anti-Ebola virus glycoprotein antibody as described in Table 3. In particular embodiments, the method comprises administering to the individual an effective amount of anti-Ebola virus glycoprotein antibody h13C6i, an effective amount of anti-Ebola virus glycoprotein antibody h2G4n, and an effective amount of anti-Ebola virus glycoprotein antibody h4G7b. In another such embodiment, the method further comprises administering to the individual an effective amount of at least one additional therapeutic agent, e.g., as described below. An “individual” according to any of the above embodiments is preferably a human.

In a further aspect, the invention provides pharmaceutical formulations comprising any of the anti-Ebola virus glycoprotein antibodies provided herein, e.g., for use in any of the above therapeutic methods. In one embodiment, a pharmaceutical formulation comprises any of the anti-Ebola virus glycoprotein antibodies provided herein and a pharmaceutically acceptable carrier. In another embodiment, a pharmaceutical formulation comprises any of the anti-Ebola virus glycoprotein antibodies provided herein and at least one additional therapeutic agent, e.g., as described below.

Antibodies of the invention can be used either alone or in combination with other agents in a therapy. For instance, an antibody of the invention may be co-administered with at least one additional therapeutic agent. In certain embodiments, additional therapeutic agents include but are not limited to: cAd3-EBOZ, rVSV-EBOV, VRC-EBODNA023-00-VP, VRC-MARDNA025-00-VP, TKM-Ebola, BCX4430, phosphorodiamidate morpholino oligomers, favipiravir, and brincidofovir. In other embodiments, prophylactic treatment and/or therapeutic treatment can include supportive care in addition to administration of the antibodies of the invention and additional therapeutic agents. Examples of supportive care include but are not limited to: preventing intravascular volume depletion, correcting profound electrolyte abnormalities, avoiding complications of shock, fluid and electrolyte replacement, respiratory support, blood transfusions, administration of antipyretic agents, analgesic agents to manage pain, antiemetic medications to control nausea and vomiting, anti-motility agents to control diarrhea, dialysis, or total renal replacement therapy.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-Ebola virus glycoprotein antibody and administration of an additional therapeutic agent occur within about one month, or within about one, two or three weeks, or within about one, two, three, four, five, or six days, of each other.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to about 45 mg/kg (e.g., about 1.0 mg/kg to about 15 mg/kg) of antibody can be an initial candidate dosage for administration to the patient, whether, for example, by one or more separate administrations, or by continuous infusion. One typical daily dosage might range from about 1 μg/kg to about 100-150 mg/kg or more, depending on the factors mentioned above. For repeated administrations over several days or longer, depending on the condition, the treatment would generally be sustained until a desired suppression of disease symptoms occurs. Exemplary dosages of the antibody would be in the range from about 1.0 mg/kg to about 150 mg/kg, from about 1.0 mg/kg to about 125 mg/kg, from about 1.0 mg/kg to about 100 mg/kg, from about 1.0 mg/kg to about 75 mg/kg, from about 1.0 mg/kg to about 45 mg/kg, from about 1.0 mg/kg to about 30 mg/kg, from about 1.0 mg/kg to about 15 mg/kg, from about 1.0 mg/kg to about 10 mg/kg, or from about 1.0 mg/kg to about 5 mg/kg. Thus, one or more doses of about 1.0 mg/kg, 2.5 mg/kg, 5.0 mg/kg, 10 mg/kg, 15 mg/kg, 30 mg/kg, 45 mg/kg, 75 mg/kg, 100 mg/kg, 125 mg/kg, or 150 mg/kg (or any combination thereof) may be administered to the patient. Such doses may be administered intermittently, e.g., every day, every two days, every three days, etc. An initial higher loading dose, followed by one or more lower doses may be administered. Dosing can also be at a fixed dose, such as, for example, 200 mg, 400 mg, 600 mg, 800 mg, 1000 mg, 1200 mg, 1400 mg, 1500 mg, 1600 mg, 1800 mg, 2000 mg, 2200 mg, 2400 mg, 2500 mg, 2600 mg, 2800 mg, 3000 mg, 3200 mg, 3400 mg, 3600 mg, 5,000 mg, 6,400 mg, 8,400 mg, 10,400 mg, etc. The progress of this therapy is easily monitored by conventional techniques and assays.

It is understood that any of the above formulations or therapeutic methods may be carried out using an immunoconjugate of the invention in place of or in addition to an anti-Ebola virus glycoprotein antibody.

H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-Ebola virus glycoprotein antibody.

### III. Examples

The following are examples of methods and compositions of the invention. It is understood that various other embodiments may be practiced, given the general description provided above.

### Reagents

ZMapp chimeric monoclonal antibodies, c13C6, c2G4, and c4G7, produced in tobacco plants, were supplied by Larry Zeitlin (Mapp Biopharmaceutical, San Diego, Calif.).

### Example 1: Humanization of Anti-EBOV Chimeric Monoclonal Antibodies c13C6, c2G4, and c4G7

Each of the three chimeric monoclonal anti-EBOV antibodies, c13C6, c2G4, and c4G7, were humanized as described below. 13C6 was humanized to kappa1/VH2, 2G4 was humanized to kappa1/VH3, and 4G7 was humanized to kappa1/VH1. Variable region amino acid sequences of each of the chimeric anti-EBOV antibodies c13C6, c2G4, and c4G7 (obtained from U.S. Pat. No. 8,513,391 and U.S. Pat. No. 7,335,356) were aligned to the closest human germline sequences for both heavy and light chains. Hypervariable regions were engineered into light and heavy chain acceptor frameworks to generate humanized CDR grafts along with additional variants that included various combinations of one or more mouse Vernier positions. Humanized nucleic acid constructs corresponding to the amino acid sequences of the various humanized versions of the three anti-EBOV monoclonal antibodies were produced by gene synthesis using standard gene synthesis methodologies available and known to one of skill in the art.

### Example 2: Humanized Anti-EBOV h13C6 Monoclonal Antibody

Twelve humanized variants of monoclonal antibody 13C6 were designed using the method described above in Example 1. The humanized variants of monoclonal antibody 13C6 and their associated designations are shown in Table 1 above along with the sequences for VH and VL and also shown below in Table 5.

TABLE 5
Humanized 13C6 antibody variants

| Vernier positions | K1 | K1 + 43 | K1 + 43, 87 |
|---|---|---|---|
| VH2 | h13C6a | h13C6e | h13C6i |
| VH2 + 2, 24, 37 | h13C6d | h13C6h | h13C6l |
| VH2 + 2, 24, 37, 73, 75 | h13C6c | h13C6g | h13C6k |
| VH2 + 2, 24, 37, 66, 68, 73, 75 | h13C6b | h13C6f | h13C6j |

In order to compare expression, affinity, activity, and FcγRIIIa binding, each of the 12 humanized variants of monoclonal antibody 13C6 were expressed as Fab fragments and as full-length IgG (FL) antibodies in 293T cells, CHO cells, and FUT8KO CHO cells. FUT8KO CHO cells contain a deletion in the FUT8 gene which results in no fucose addition to expressed proteins (i.e., afucosylated proteins). Small-scale (30m1) expression experiments were performed for each monoclonal antibody variant.

Each of the expressed monoclonal antibodies was purified by affinity chromatography using MabSelect SuRe (GE Healthcare, 17-5438). Eluted material was buffer exchanged into buffer containing 10 mM Histidine, 240 mM Sucrose, and 0.01% tween 20.

The humanized variants of monoclonal antibody 13C6 expressed well, with an average yield of approximately 1.3 mg/30m1 culture, and 97.2% monomer by analytical HPLC-SEC.

Each of the humanized variants of monoclonal antibody 13C6 (Fab or FL) produced in 293T cells, CHO cells, or FUT8KO CHO cells was examined for non-specific binding to baculovirus particles by ELISA according to previously published methods (Hotzel, et al. (2012) MAbs. 4: 753-760.) All humanized variants of monoclonal antibody 13C6 displayed very low non-specific binding to baculovirus particles in this assay.

Binding affinity to EBOV antigen for each humanized variant of monoclonal antibody 13C6 was determined using a Biacore T200 instrument as follows. Recombinant EBOV GPd™ (recombinant EBOV GPd™, IBT Bioservices, cat.#0501-015, Lot: 1411003) was immobilized on a Biacore Series S CM5 sensor chip (GE Healthcare) at low desity (200 RU), medium density (600 RU), and high density (2000 RU). A concentration series of the anti-EBOV monoclonal antibodies, ranging from 4.11-333 nM, was flowed over the chip. Interaction of each humanized anti-EBOV monoclonal antibody variant was analyzed by single cycle kinetics with minimal regeneration to preserve the integrity of immobilized antigen. A flow rate of 30 μl/min was used with a 5-minute dissociation for each cycle of the assay. A 1:1 Langmuir binding model with simultaneous fitting of kₒₙ and k_{off} was applied for kinetic analysis. Initial affinity was determined for variants expressed in FUT8KO. Table 6 provides initial characterization of humanized 13C6 antibody variants and Biacore affinity summary for the antibody variants expressed in FUT8KO.

TABLE 6

| Chimeric 13C6 = 14 nM | K1 | K1 + 43 | K1 + 43, 87 |
|---|---|---|---|
| VH2 | 14 nM | 18 nM | 12 nM |
| | (h13C6a) | (h13C6e) | (h13C6i) |
| VH2 + 2, 24, 37 | 85 nM | 67 nM | 35 nM |
| | (h13C6d) | (h13C6h) | (h13C6l) |
| VH2 + 2, 24, 37, 73, 75 | 57 nM | 52 nM | 19 nM |
| | (h13C6c) | (h13C6g) | (h13C6k) |
| VH2 + 2, 24, 37, 66, 68, 73, 75 | 40 nM | 42 nM | 23 nM |
| | (h13C6b) | (h13C6f) | (h13C6j) |

The highest affinity antibody variants (i.e., h13C6a, h13C6e, and h13C6i) were re-assayed by Biacore as described above comparing affinities of the antibody variants with commercial chimeric c13C6 from IBT Bioservices. Table 7 and Table 8 provide Biacore affinities for hu13C6a, hu13C6e, and hu13C6i expressed in CHO or FUT8KO to low density recombinant EBOV GP and high density recombinant EBOV GP, respectively.

TABLE 7
Low Density recombinant EBOV GP coat (200 RU)

| mAb Variant | Kon | Koff | KD (nM) | RU max |
|---|---|---|---|---|
| CHO (h13C6a) | 2.10e4 | 8.4e−4 | 40 | 204 |
| CHO (h13C6e) | 1.44e4 | 8.21e−4 | 57 | 97 |
| CHO (h13C6i) | 2.05e4 | 5.54e−4 | 27 | 111 |
| FKO (h13C6a) | 2.28e4 | 5.70e−4 | 25 | 121 |
| FKO (h13C6e) | 1.54e4 | 4.15e−4 | 27 | 150 |
| FKO (h13C6i) | 1.74e4 | 3.24e−4 | 12 | 194 |
| c13C6 (IBT) | 1.46e4 | 2.04e−4 | 14 | 270 |

TABLE 8
High Density recombinant EBOV GP coat (2000 RU)

| mAb Variant | Kon | Koff | KD (nM) | RU max |
|---|---|---|---|---|
| CHO (h13C6a) | 1.96e4 | 4.5e−4 | 23 | 221 |
| CHO (h13C6e) | 1.49e4 | 6.27e−4 | 42 | 112 |
| CHO (h13C6i) | 1.74e4 | 3.31e−4 | 19 | 165 |
| FKO (h13C6a) | 1.78e4 | 3.11e−4 | 17 | 240 |
| FKO (h13C6e) | 1.71e4 | 3.29e−4 | 19 | 225 |
| FKO (h13C6i) | 1.58e4 | 1.96e−4 | 13 | 141 |
| c13C6 (IBT) | 1.47e4 | 1.48e−4 | 10 | 306 |

Humanized 13C6 variant h13C6i and humanized variant h13C6a were identified as good binders. An additional Biacore binding analysis was done to assess binding to recombinant FcγRIIIa (R&D Systems, cat#4325-FC-050). Expression in FUT8KO cells improved FcγRIIIa (V158) binding about 3-fold compared with CHO cell expressed material as shown in Table 9.

TABLE 9

| | Low density | High density |
|---|---|---|
| Reagent | KD (nM) | KD (nM) |
| Herceptin CHO | 82 | 67 |
| h13C6a CHO | 117 | 113 |
| h13C6i CHO | 122 | 111 |
| h13C6a FUT8KO | 38 | 27 |
| h13C6i FUT8KO | 36 | 24 |
| Commercial c13C6 | 44 | 32 |

Additional in vitro analysis for humanized variant h13C6i is summarized in Table 10.

TABLE 10

| Characterization | c13C6 tobacco | h13C6I transient |
|---|---|---|
| Biacore KD | 10 nM | 12 nM |
| Aggregation | 14-20% | <0.3% |
| Mannose | 16% | ~4% |
| Fucosylation | 0%, incomplete glycosylation | 0%, normal |
| | present | glycosylation |
| Mixed disulfides | 0.5 moles/mole of mAb | <0.1 moles/mole |
| and free thiol | | of mAb |
| Met 257 oxidation | ~20% | ~4% |
| Temp dependent | 15% increase in aggregations | None observed |
| aggregation | upon chilling (may be NaCl | |
| | dependent) | |
| FcγRIIIa (V158) | 32 nM | 24 nM |
| binding | | |

### Example 3: Humanized Anti-EBOV h2G4 Monoclonal Antibody

Twenty humanized variants of monoclonal antibody 2G4 were designed and sixteen were made using the method described above in Example 1. The humanized variants of monoclonal antibody 2G4 and their associated designations are shown in Table 2 above along with the sequences for VH and VL and also shown below in Table 11.

TABLE 11
Humanized 2G4 antibody variants

| | | | | | K1 + 43, |
|---|---|---|---|---|---|
| Vernier positions | K1 | K1 + 43 | K1 + 48 | K1 + 43, 48 | 48, 71 |
| VH3 | h2G4a | h2G4e | h2G4i | hu2G4q* | hu2G4n |
| VH3 + 49 | h2G4b | h2G4f | h2G4j | hu2G4r* | hu2G4o |
| VH3 + 78 | h2G4c | h2G4g | h2G4k | hu2G4s* | hu2G4t* |
| VH3 + 49, 78 | h2G4d | h2G4h | h2G4l | hu2G4m | hu2G4p |

*variant not yet made

The sixteen humanized 2G4 variant antibodies were expressed as full length IgG in 293T cells at small-scale 30m1 to test antigen binding. Initial affinity measurements were done using a Biacore T200 instrument, as described above for humanized 13C6 variant antibodies. For the humanized 2G4 variant antibodies, a higher concentration series than that described for 13C6 was necessary to determine Kd affinity based on the reported affinity for the chimeric 2G4 version in the 200-500 nM range. The concentration range applied was between 1.8 μM-22.2 nM. The results of the initial affinity measurements are provided in Table 12.

TABLE 12

| Chimeric 2G4 = | | | | | K1 + 43, |
|---|---|---|---|---|---|
| 427 nM | K1 | K1 + 43 | K1 + 48 | K1 + 43, 48 | 48, 71 |
| VH3 | 127 nM | 112 nM | 211 nM | ND | 88 nM |
| | (h2G4a) | (h2G4e) | (h2G4i) | (hu2G4q) | (hu2G4n) |
| VH3 + 49 | 160 nM | 147 nM | 214 nM | ND | 152 nM |
| | (h2G4b) | (h2G4f) | (h2G4j) | (hu2G4r) | (hu2G4o) |
| VH3 + 78 | 130 nM | 197 nM | 213 nM | ND | ND |
| | (h2G4c) | (h2G4g) | (h2G4k) | (hu2G4s) | (hu2G4t) |
| VH3 + 49, 78 | 129 nM | 111 nM | 171 nM | 188 nM | 154 nM |
| | (h2G4d) | (h2G4h) | (h2G4l) | (hu2G4m) | (hu2G4p) |

Humanized candidate, h2G4n, was identified from all of the humanized 2G4 variant antibodies assayed based on having the highest affinity and cleanest BV ELISA score. This candidate, h2G4n, was expressed at larger scale with additional purification steps and re-assayed for binding to recombinant antigen. Binding assays were done comparing chimeric material produced in tobacco plants. Meaningful kinetic data were not determined for tobacco produced chimeric 2G4 antibody due to high non-specific binding to a blank flowcell, possibly a result of high aggregate content in the chimeric preps. Published affinity values for chimeric 2G4 antibody are included in Tables 12 and 13 for comparison. Table 13 provides the results of the binding assays. As seen in Table 13, for 2G4, small scale in-house chimera (293T) was about 3.4 times better than published affinity. Small scale humanized 2G4N (293T) had similar affinity. Large scale pooled material, PUR 78032 is ˜7 times and the SEC pool is ˜9 times better than published affinity.

TABLE 13

| | | | KD (nM) | KD (nM) |
|---|---|---|---|---|
| | | | high | low |
| Variant | VL | VH | density | density |
| 2G4 commercial | chimera | chimera | ND | ND |
| 2G4 published | chimera | chimera | 427 | ND |
| 2G4 in-house | chimera | chimera | 123 | ND |
| h2G4n (30 ml) | K1 + 43, 48, 71 | H3 | 88 | ND |
| h2G4n PUR 78031 | K1 + 43, 48, 71 | H3 | 12 | 26 |
| Final pool | | | | |
| h2G4n PUR 78495 | K1 + 43, 48, 71 | H3 | 9 | 18 |
| SEC pool | | | | |

### Example 4: Humanized Anti-EBOV h4G7 Monoclonal Antibody

Fifteen humanized variants of monoclonal antibody 4G7 were designed and made using the method described above in Example 1. The humanized variants of monoclonal antibody 4G7 and their associated designations are shown in Table 3 above along with the sequences for VH and VL and also shown below in Table 14.

TABLE 14
Humanized 4G7 antibody variants

| | | | | K1 + 43, | |
|---|---|---|---|---|---|
| Vernier positions | K1 | K1 + 43 | K1 + 43, 48 | 48, 87 | K1 + 48 |
| VH1 | h4G7b | h4G7e | h4G7h | h4G7k | h4G7n |
| VH1 + 67, 69, 71 | h4G7c | h4G7f | h4G7i | h4G7l | h4G7o |
| VH1 + 71 | h4G7d | h4G7g | h4G7j | h4G7m | h4G7p |

The fifteen humanized 4G7 variant antibodies were expressed as full length IgG in 293T cells at small-scale 30m1 to test antigen binding. Initial affinity measurements were done using a Biacore T200 instrument, as described above for humanized 13C6 variant antibodies. For the humanized 4G7 variant antibodies, a higher concentration series than that described for 13C6 was necessary to determine Kd affinity based on the reported affinity for the chimeric 4G7 version in the 200-500 nM range. The concentration range applied was between 1.8 μM-22.2 nM. The results of the initial affinity measurements are provided in Table 15.

TABLE 15

| Chimeric 4G7 = | | | | K1 + 43, | |
|---|---|---|---|---|---|
| 215 nM | K1 | K1 + 43 | K1 + 43, 48 | 48, 87 | K1 + 48 |
| VH1 | 155 nM | 215 nM | 220 nM | 177 nM | 164 nM |
| | (h4G7b) | (h4G7e) | (h4G7h) | (h4G7k) | (h4G7n) |
| VH1 + 67, 69, 71 | 215 nM | 311 nM | 357 nM | 412 nM | 324 nM |
| | (h4G7c) | (h4G7f) | (h4G7i) | (h4G7l) | (h4G7o) |
| VH1 + 71 | 171 nM | TBD | TBD | TBD | TBD |
| | (h4G7d) | (h4G7g) | (h4G7j) | (h4G7m) | (h4G7p) |

Humanized candidate, h4G7b, was identified from all of the humanized 4G7 variant antibodies assayed based on having the highest affinity and cleanest BV ELISA score. This candidate, h4G7b, was expressed at larger scale with additional purification steps and re-assayed for binding to recombinant antigen. Binding assays were done comparing chimeric material produced in tobacco plants. Meaningful kinetic data were not determined for tobacco produced chimeric 4G7 antibody due to high non-specific binding to a blank flowcell, possibly a result of high aggregate content in the chimeric preps. Published affinity values for chimeric 4G7 antibody are included in Tables 15 and 16 for comparison. Table 16 provides the results of the binding assays. As seen in Table 13, for 4G7, small scale in-house chimera (293T) appeared to be somewhat better than published affinity. Small scale humanized 4G7b (293T) had similar affinity. Large scale pooled 4G7b PUR 78032 is ˜1.5 times better.

TABLE 16

| | | | KD (nM) | KD (nM) |
|---|---|---|---|---|
| Variant | VL | VH | high density | low density |
| 4G7 commercial | chimera | chimera | ND | ND |
| 4G7 published | chimera | chimera | 215 | ND |
| 4G7 in-house | chimera | chimera | 140 | ND |
| h4G7b (small scale | K1 | H1 | 155 | ND |
| 30 ml) | | | | |
| h4G7b PUR 78032 | K1 | H1 | 102 | 112 |
| final pool | | | | |
| h4G7b SEC | K1 | H1 | Not | Not |
| | | | determined | determined |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An isolated, humanized antibody that binds to Ebola virus glycoprotein.

2. The antibody of claim 1, wherein the antibody binds to an epitope comprising the amino acid sequence of SEQ ID NO:91.

3. The antibody of claim 1, wherein the antibody binds to an epitope comprising the amino acid sequence of SEQ ID NO:92.

4. The antibody of claim 1, wherein the antibody comprises (a) HVR-H1 comprising the amino acid sequence of SEQ ID NO:22; (b) HVR-H2 comprising the amino acid sequence of SEQ ID NO:23; and (c) HVR-H3 comprising the amino acid sequence of SEQ ID NO:24.

5. The antibody of claim 1, wherein the antibody comprises (a) HVR-L1 comprising the amino acid sequence of SEQ ID NO:9; (b) HVR-L2 comprising the amino acid sequence of SEQ ID NO:10; and (c) HVR-L3 comprising the amino acid sequence of SEQ ID NO:11.

6. The antibody of claim 1, wherein the antibody comprises (a) HVR-H1 comprising the amino acid sequence of SEQ ID NO:22; (b) HVR-H2 comprising the amino acid sequence of SEQ ID NO:23; (c) HVR-H3 comprising the amino acid sequence of SEQ ID NO:24; (d) HVR-L1 comprising the amino acid sequence of SEQ ID NO:9; (e) HVR-L2 comprising the amino acid sequence of SEQ ID NO:10; and (f) HVR-L3 comprising the amino acid sequence of SEQ ID NO:11.

7. The antibody of claim 1, wherein the antibody comprises a heavy chain variable region, wherein the heavy chain variable region comprises an amino acid sequence selected from the group consisting of SEQ ID NOs:14, 16, 18, and 20.

8. The antibody of claim 1, wherein the antibody comprises a light chain variable region, wherein the light chain variable region comprises an amino acid sequence selected from the group consisting of SEQ ID NOs:3, 5, and 7.

9. The antibody of claim 1, wherein the antibody comprises a heavy chain variable region and a light chain variable region, wherein the heavy chain variable region comprises an amino acid sequence selected from the group consisting of SEQ ID NOs:14, 16, 18, and 20, and wherein the light chain variable region comprises an amino acid sequence selected from the group consisting of SEQ ID NOs:3, 5, and 7.

10. The antibody of claim 1, wherein the antibody comprises a heavy chain, wherein the heavy chain comprises an amino acid sequence selected from the group consisting of SEQ ID NOs:15, 17, 19, and 21.

11. The antibody of claim 1, wherein the antibody comprises a light chain, wherein the light chain comprises an amino acid sequence selected from the group consisting of SEQ ID NOs:4, 6, and 8.

12. The antibody of claim 1, wherein the antibody comprises a heavy chain and a light chain, wherein the heavy chain comprises an amino acid sequence selected from the group consisting of SEQ ID NOs:15, 17, 19, and 21, and wherein the light chain comprises an amino acid sequence selected from the group consisting of SEQ ID NOs:4, 6, and 8.

13. The antibody of claim 1, wherein the antibody comprises (a) HVR-H1 comprising the amino acid sequence of SEQ ID NO:50; (b) HVR-H2 comprising the amino acid sequence of SEQ ID NO:51; and (c) HVR-H3 comprising the amino acid sequence of SEQ ID NO:52.

14. The antibody of claim 1, wherein the antibody comprises (a) HVR-L1 comprising the amino acid sequence of SEQ ID NO:37; (b) HVR-L2 comprising the amino acid sequence of SEQ ID NO:38; and (c) HVR-L3 comprising the amino acid sequence of SEQ ID NO:39.

15. The antibody of claim 1, wherein the antibody comprises (a) HVR-H1 comprising the amino acid sequence of SEQ ID NO:50; (b) HVR-H2 comprising the amino acid sequence of SEQ ID NO:51; (c) HVR-H3 comprising the amino acid sequence of SEQ ID NO:52; (d) HVR-L1 comprising the amino acid sequence of SEQ ID NO:37; (e) HVR-L2 comprising the amino acid sequence of SEQ ID NO:38; and (f) HVR-L3 comprising the amino acid sequence of SEQ ID NO:39.

16. The antibody of claim 1, wherein the antibody comprises a heavy chain variable region, wherein the heavy chain variable region comprises an amino acid sequence selected from the group consisting of SEQ ID NOs:42, 44, 46, and 48.

17. The antibody of claim 1, wherein the antibody comprises a light chain variable region, wherein the light chain variable region comprises an amino acid sequence selected from the group consisting of SEQ ID NOs:27, 29, 31, 33, and 35.

18. The antibody of claim 1, wherein the antibody comprises a heavy chain variable region and a light chain variable region, wherein the heavy chain variable region comprises an amino acid sequence selected from the group consisting of SEQ ID NOs: 42, 44, 46, and 48, and wherein the light chain variable region comprises an amino acid sequence selected from the group consisting of SEQ ID NOs: 27, 29, 31, 33, and 35.

19. The antibody of claim 1, wherein the antibody comprises a heavy chain, wherein the heavy chain comprises an amino acid sequence selected from the group consisting of SEQ ID NOs:43, 45, 47, and 49.

20. The antibody of claim 1, wherein the antibody comprises a light chain, wherein the light chain comprises an amino acid sequence selected from the group consisting of SEQ ID NOs:28, 30, 32, 34, and 36.

21. The antibody of claim 1, wherein the antibody comprises a heavy chain and a light chain, wherein the heavy chain comprises an amino acid sequence selected from the group consisting of SEQ ID NOs:43, 45, 47, and 49, and wherein the light chain comprises an amino acid sequence selected from the group consisting of SEQ ID NOs:28, 30, 32, 34, and 36.

22. The antibody of claim 1, wherein the antibody comprises (a) HVR-H1 comprising the amino acid sequence of SEQ ID NO:76; (b) HVR-H2 comprising the amino acid sequence of SEQ ID NO:77; and (c) HVR-H3 comprising the amino acid sequence of SEQ ID NO:78.

23. The antibody of claim 1, wherein the antibody comprises (a) HVR-L1 comprising the amino acid sequence of SEQ ID NO:65; (b) HVR-L2 comprising the amino acid sequence of SEQ ID NO:66; and (c) HVR-L3 comprising the amino acid sequence of SEQ ID NO:67.

24. The antibody of claim 1, wherein the antibody comprises (a) HVR-H1 comprising the amino acid sequence of SEQ ID NO:76; (b) HVR-H2 comprising the amino acid sequence of SEQ ID NO:77; (c) HVR-H3 comprising the amino acid sequence of SEQ ID NO:78; (d) HVR-L1 comprising the amino acid sequence of SEQ ID NO:65; (e) HVR-L2 comprising the amino acid sequence of SEQ ID NO:66; and (f) HVR-L3 comprising the amino acid sequence of SEQ ID NO:67.

25. The antibody of claim 1, wherein the antibody comprises a heavy chain variable region, wherein the heavy chain variable region comprises an amino acid sequence selected from the group consisting of SEQ ID NOs:70, 72, and 74.

26. The antibody of claim 1, wherein the antibody comprises a light chain variable region, wherein the light chain variable region comprises an amino acid sequence selected from the group consisting of SEQ ID NOs:55, 57, 59, 61, and 63.

27. The antibody of claim 1, wherein the antibody comprises a heavy chain variable region and a light chain variable region, wherein the heavy chain variable region comprises an amino acid sequence selected from the group consisting of SEQ ID NOs:70, 72, and 74, and wherein the light chain variable region comprises an amino acid sequence selected from the group consisting of SEQ ID NOs:55, 57, 59, 61, and 63.

28. The antibody of claim 1, wherein the antibody comprises a heavy chain, wherein the heavy chain comprises an amino acid sequence selected from the group consisting of SEQ ID NOs:71, 73, and 75.

29. The antibody of claim 1, wherein the antibody comprises a light chain, wherein the light chain comprises an amino acid sequence selected from the group consisting of SEQ ID NOs:56, 58, 60, 62, and 64.

30. The antibody of claim 1, wherein the antibody comprises a heavy chain and a light chain, wherein the heavy chain comprises an amino acid sequence selected from the group consisting of SEQ ID NOs: 71, 73, and 75, and wherein the light chain comprises an amino acid sequence selected from the group consisting of SEQ ID NOs:56, 58, 60, 62, and 64.

31. The antibody of claim 1, wherein the antibody comprises (a) a heavy chain variable region amino acid sequence having at least 90% amino acid sequence identity to the amino acid sequence of SEQ ID NO:14, (b) a light chain, variable region amino acid sequence having at least 90% amino acid sequence identity to the amino acid sequence of SEQ ID NO:7, or (c) a heavy chain variable region amino acid sequence region as in (a) and a light chain variable region amino acid sequence as in (b).

32. The antibody of claim 1, wherein the antibody comprises (a) a heavy chain variable region amino acid sequence having at least 90% amino acid sequence identity to the amino acid sequence of SEQ ID NO:42, (b) a light chain variable region amino acid sequence having at least 90% amino acid sequence identity to the amino acid sequence of SEQ ID NO:35, or (c) a heavy chain variable region amino acid sequence region as in (a) and a light chain variable region amino acid sequence as in (b).

33. The antibody of claim 1, wherein the antibody comprises (a) a heavy chain variable region amino acid sequence having at least 90% amino acid sequence identity to the amino acid sequence of SEQ ID NO:70, (b) a light chain variable region amino acid sequence having at least 90% amino acid sequence identity to the amino acid sequence of SEQ ID NO:55, or (c) a heavy chain variable region amino acid sequence region as in (a) and a light chain variable region amino acid sequence as in (b).

34. The antibody of claim 1, wherein the antibody binds to an Ebola virus glycoprotein comprising a sequence selected from the group consisting of SEQ ID NOS:79-90.

35. The antibody of claim 1, which is an antibody fragment.

36. The antibody of claim 1, which is a full length IgG antibody.

37. The antibody of claim 1, wherein the antibody is afucosylated.

38. An isolated nucleic acid molecule encoding the antibody of claim 1.

39. A host cell comprising the nucleic acid molecule of claim 38.

40. The host cell of claim 39, wherein the host cell is a host cell that is capable of producing afucosylated antibodies.

41. The host cell of claim 39, wherein the host cell is a mammalian host cell that is capable of producing afucosylated antibodies.

42. The host cell of claim 40, wherein the host cell is a mammalian host cell that has a deletion in the FUT8 gene which results in reduced or no fucose addition to expressed proteins.

43. A method of producing an antibody comprising culturing the host cell of claim 39.

44. The method of claim 43, wherein the method results in decreased antibody aggregation, relative to producing the antibody in a host cell that does not produce afucosylated antibodies.

45. An immunoconjugate comprising the antibody of claim 1 and a cytotoxic agent.

46. A pharmaceutical formulation comprising the antibody of claim 1 or the immunoconjugate of claim 45, and a pharmaceutically acceptable carrier or diluent.

47. The pharmaceutical formulation of claim 46, further comprising an additional therapeutic agent.

48. The pharmaceutical formulation of claim 47, wherein the additional therapeutic agent is another anti-Ebola virus antibody, an anti-viral agent, or an anti-Ebola vaccine.

49. (canceled)

50. (canceled)

51. (canceled)

52. A method of treating an individual having Ebola virus infection comprising administering to the individual an effective amount of the antibody claim 1 or the immunoconjugate of claim 45.

53. The method of claim 52, further comprising administering an additional therapeutic agent to the individual.

54. The method of claim 53, wherein the additional therapeutic agent is another anti-Ebola virus antibody, an anti-viral agent, or an anti-Ebola vaccine.
